SEQUENCE LISTING (8 sequences)

SEQ ID NO: 1 - DNA, 1200 bp, Bacillus paralicheniformis
  source 1..1200: mol_type = genomic DNA
atgagggtattcgttgccagacagccaatattcaacagaaaagaacaagttgtcgcatacgagcttttatacagagaaagcgagaaaaactttttttccggtattgacggtgatcaagcgacaacagaattgatgattaacagctttttaaacattggaatcgataaattgacagaaggcaaaaggtattacgtgaattttaccgaaggtcttttggcatcagggctgccgacttattttgatccggaccagctcgttgtagaaatccttgaagatgtgccgatcacccctgagcttatcgaaaggtgcaggcatttgaaaagcctcggctacacgattgcccttgatgatttttgtttaaaacatcgcgtggagagggacttgctccatcagcttttggcatccattgatattttgaagatcgattttttcaaaacgacgcggcaggaaagacagagcatccttcgatcatgccggaaccaccgtctgacgtttctcgctgaaaaagtggagacgcgaaaggattatgagcaggcggcaaaagacggatttcaactgtttcaaggctatttcttcagcgagcccgctgtgattgacggtcaggacattacatatcatttccatgcttactatgagctgctgcatgaattaagcgaagatcagcctgatattgaaaacgtcacaaatattatagaacgcgatttgtcgctttcctaccagctgctgaagctgttgaactctccggccaaccggccgattcaaaagattaaaagcatccgccaggctatcgtgcttctcggatttaaagaaatcaaaaggtggatcttcatcctctcatttaaggatttaacgaaaaaacaaaactccagcaagaacgaagtcgtcaagatttcgctgatccgcgcgaagctttgcgaattgctggcgaaaaaaacgaatcgccctcagccggcttcttatatgctgacaggcatgttttcatttatcgatacactgcttcacaaagaactggcggaagtcatcagcgaactgccgctgacggacgaagtaggacaagcactgctcggcaaagaaaacgattatcgaaaaatcttacggcttgcaaaatcgatcgagcgaaacgaatgggaagacagcacgccagaaacagaaggcttaacaaaagatgaagcgtatcaatgctatcttgaagccgttgat

SEQ ID NO: 2 - protein, 406 aa, Bacillus paralicheniformis
MRVFVARQPIFNRKEQVVAYELLYRESEKNFFSGIDGDQATTELMINSFLNIGIDKLTEGKRYYVNFTEGLLASGLPTYFDPDQLVVEILEDVPITPELIERCRHLKSLGYTIALDDFCLKHRVERDLLHQLLASIDILKIDFFKTTRQERQSILRSCRNHRLTFLAEKVETRKDYEQAAKDGFQLFQGYFFSEPAVIDGQDITYHFHAYYELLHELSEDQPDIENVTNIIERDLSLSYQLLKLLNSPANRPIQKIKSIRQAIVLLGFKEIKRWIFILSFKDLTKKQNSSKNEVVKISLIRAKLCELLAKKTNRPQPASYMLTGMFSFIDTLLHKELAEVISELPLTDEVGQALLGKENDYRKILRLAKSIERNEWEDSTPETEGLTKDEAYQCYLEAVDWCQKLL

SEQ ID NO: 3 - DNA, 1644 bp, Bacillus licheniformis
  source 1..1644: mol_type = genomic DNA
atgaataaacgcaaaacaggattttcaattttaagcttgctgctgatcttatcgatttttctaacggcctgcaacagcggcgaagtcggaggggacgaaaaggaaggcaaatctgacgggaagccgcagcagggcggggatctgattgctggatccacaggtgaaccgacgctgtttaattcactgtattcaactgatacagcaagttcagatattgaaagactgatctacaacactttgttagacgttaatgagaaacttgaagtggagaaccagcttgctgaggaagtaaaagaatcggaagatggtttaacatttgatgttaagctcaaggaaggcgtcaagtttcatgacggtgaagaaatgacagctgatgatgttgtatttacttacagcattccgatgagtgatgactacgtcggagaacgcggctcgaactttaaaatgatagagtctgtcacgaaaaaaggtaaatatgaagtacagtttaaattaaagaagcctgatccgtatttttacaatgttacacttgccagttacggtattctgcctaagcacattttaaaagatgtcccaatcagcaaactcggtgaacacgaattcaatcgaaagaatccgattggaacaggaccgtttaaatttaaagaatggaaagaaggacaatatgtaaaggttgaggcttttgatgattattatgccgggcgtcctcatttagattcgatcacgtataaaattattccggattcaaatgcagcgctgtcacagctgcaagctggagatgtcgattacttggtcgttacaccaggaccagactataaaacggccgagaaatttaacaatgtgaagatggaaaccgatttaggtttgaattatacgtatatcggctggaacgaaagaaatgagctgtttaaggataaaaaggttcgccaagcgctgacacatgcgcttgaccgccaggcgctcgttgaccaagttctagatggagatggggaaatcgcgaacatcccggaaagcccgctttcatggaactatccggataacaaagataagtttaaaacgtttgaatacgatccagagaaagcaaaaaaactgctcaaagaagctggatggacagactcagatggtgatgggattttagataaggacggcaaaaagttttctttcgttattaaaacgaaccaaggaaacaaaacacgtgaagacctcgcggttgttgttcagcaacaattaaaagaaattggtattcaagcaaaaccgcagattgttgaatggagtgctttaattgaacaaatgaatccgccgaattgggattttgatgcgatgatcatgggctggagccttgctacgttccctgatcagagcaacattttccattcgaaagaagctgaaaaaggactaaactatgtttggtatcaaaataaaaagcttgataaattgctggatgaagctaaaacgttgaaggatcgtgaagaatataaaaaggcgtatgaagatatttacgaaattctggcggaagatcagccatatacattcttgtattatacgaactaccatagagcgatgccaaaaaatatgaaaggttacgtcttccatccgaaagaagatttctataaagccgaagactggtggctggatcaaaaataa

SEQ ID NO: 4 - protein, 547 aa, Bacillus licheniformis
MNKRKTGFSILSLLLILSIFLTACNSGEVGGDEKEGKSDGKPQQGGDLIAGSTGEPTLFNSLYSTDTASSDIERLIYNTLLDVNEKLEVENQLAEEVKESEDGLTFDVKLKEGVKFHDGEEMTADDVVFTYSIPMSDDYVGERGSNFKMIESVTKKGKYEVQFKLKKPDPYFYNVTLASYGILPKHILKDVPISKLGEHEFNRKNPIGTGPFKFKEWKEGQYVKVEAFDDYYAGRPHLDSITYKIIPDSNAALSQLQAGDVDYLVVTPGPDYKTAEKFNNVKMETDLGLNYTYIGWNERNELFKDKKVRQALTHALDRQALVDQVLDGDGEIANIPESPLSWNYPDNKDKFKTFEYDPEKAKKLLKEAGWTDSDGDGILDKDGKKFSFVIKTNQGNKTREDLAVVVQQQLKEIGIQAKPQIVEWSALIEQMNPPNWDFDAMIMGWSLATFPDQSNIFHSKEAEKGLNYVWYQNKKLDKLLDEAKTLKDREEYKKAYEDIYEILAEDQPYTFLYYTNYHRAMPKNMKGYVFHPKEDFYKAEDWWLDQK

SEQ ID NO: 5 - DNA, 1389 bp, Bacillus paralicheniformis
  source 1..1389: mol_type = genomic DNA
atgccagtcaattcgtttgacaactatccaatgtcttggaaacctgataagaaagcattgaagcgtccttattattattcgattgcgacattgcttgaagaggatatcgtaaacggttttttggcgcctgggacaaagctgcctccgcaacgggaactggcagattttcttgatttaaactttaccacgattacacgcgcctacaaactatgtgagttcaaggggctgatttatgctgtcaccggaagcggcaccttcgtcgctcctaatgctgcccgctctatcaccatttccgcagataaggtgacaaactgcattgatctcggatttgtagcctcttttgagcaaaccaacggaatggtagcggaggttgttcaaaaagctgcagataaaagctatttggagaagctaatggactacaatgacccgactggtattccgcatcaaaaaacggcagggctaaactggatggaatctttcggtattcacgcagaccaagaacatattgcgattgtttccggtgctcaaaatgcgttggccattgcattgacctcgctgtttgaccctggtgaccgcattgcaactgacctatacacgtattcgaactttattgagctggccaaaatgctccatattcaattggtacctgtttccggtgaccagtatggaatgctgccggatgaacttgaaaagcagtgctgccagacgaaaattcacggcatatttctgatgccctcatgctgtaatccgacaaccgtgatgatatcagatgttcgaaagcatgaattagtggaggccatccgcaagcatgatttgctgttgatcgaggatgatattcatgcgtttctgacggcagggattgtatcagattatcagcagccaatgtttagtttgcttccagatcagagcatatacatttgcagcacctcaaagtcgatatgctccgggttaagagttgcctatatggtgtatggggatgctttacgggaaaagatattgcagggcatttttaacatcaatgtcaaaacgtcatctttagatgcggaggtcattactgagctgattttatcaggtaaggctcatgaaatcgttgctcaaaagaaaaagcttgcacagtcggccaatgatctttatgcggcatattttcctgtaactgagcctggtgaacatcctcttagtttataccgatggcttccgattgaagagcatgctgactcatcacaattggagacggatttgaggaagcgtgggattcgggtttttcattccgaccgttttctcagcgggcagacgacgcgcgaaaaatatttgcgcattgcgctttcttctacaaattcattagatgagctgaaattagggttggatatattaaaacagtatctcggataa

SEQ ID NO: 6 - protein, 462 aa, Bacillus paralicheniformis
MPVNSFDNYPMSWKPDKKALKRPYYYSIATLLEEDIVNGFLAPGTKLPPQRELADFLDLNFTTITRAYKLCEFKGLIYAVTGSGTFVAPNAARSITISADKVTNCIDLGFVASFEQTNGMVAEVVQKAADKSYLEKLMDYNDPTGIPHQKTAGLNWMESFGIHADQEHIAIVSGAQNALAIALTSLFDPGDRIATDLYTYSNFIELAKMLHIQLVPVSGDQYGMLPDELEKQCCQTKIHGIFLMPSCCNPTTVMISDVRKHELVEAIRKHDLLLIEDDIHAFLTAGIVSDYQQPMFSLLPDQSIYICSTSKSICSGLRVAYMVYGDALREKILQGIFNINVKTSSLDAEVITELILSGKAHEIVAQKKKLAQSANDLYAAYFPVTEPGEHPLSLYRWLPIEEHADSSQLETDLRKRGIRVFHSDRFLSGQTTREKYLRIALSSTNSLDELKLGLDILKQYLG

SEQ ID NO: 7 - DNA, 1626 bp, Bacillus paralicheniformis
  source 1..1626: mol_type = genomic DNA
ttgaagaagcgtttgtcatttatcagtttaatgctcattttcacactcgtcctcagcgcctgcggcttcggctcaagctccggtgacggcggtaaaaaagacagcaaagggaaagacacattaaatgtcaacattaaaacagaaccgttttcactacatccgggactcgcaaacgattcggtgtctgcaaacgtgcttcgtcagacttttgaaggattgacgacaatcggtaaagatggaaagccggttgaagcggcagccgaaaaaatcgaagtcagcgacgaccaaaaaacatacacattcacgctccgcgacgcgaaatggtcaaatggagatcctgtaacagcagaggattttgaatacgcatggaaatgggcgctcgaccctaaaaacgaatcgcaatatgcgtatcagctttactacttaaaaggcggagaagcagcgaacaccggcaaagggaaaattgaagatgtcggcgttaaagctgttaatgataagactttaaaagtcgagcttgaaaaaccgacaccgtattttactgaactgacagcattctacacatatatgccagtcaataaaaaggtagcagagaagaatgcgaaatggtacacaaacgcagatgagaactacgtatctaacggacctttcaaaatggcgaaatggaagcacagcggaaacatcgtactggagaaaaacgaccagtactgggataaagacgctgttaagctgaagaaaatcaatatggcgatggtcaacgatccgaacactggtctgaacatgtacaaaaaaggcgagctagactttgtaggacagccgcttgaccagatttcaacggatgcgattccaagcctgaaaaaagaaggcctgaacattgatccgttcgcatcggtttacctgtacaaattcaacactgaagcggctccgctgaacaatgtcaacatccgtaaggcgctgacatacgcgatcaaccgcgaagcgatcgtcaaaaacatcacgcaagcggaacagctgcctgcgatgggattagtgccgccggcagtccacggctttgagtcaaataaaggctatttcaaagaccatgatgttgataaggcgaaagaatacttggaaaaaggtttgaaagagctcggtttgaaaaaagcgtcagaccttccgaaaatcacgctttccttcaacacggatgaagcacaccaaaaaatcgctcaagccgttcaggaaatgtggaacaaggaactgggcttagatgtcgaattaggcaatgaagaatggaacgtatacatcgataagctccatgcaggaaactatcaaatcggccgtttaggctggaccgccgactttaacgacggaatgaacttccttgaaacataccgcgacaaagaaggcggaaacaatgatacgaactgggaaaacgcaaagtataaagagctgctgaataaagcatcgagagaaactgattcagcgaaacgcatcgagctgatgaaagaagcagaaagcatcatcatggatgagctgccggttgcaccgatctacttctacacaatgccgtatcttcatgatgaaagcttaaaagactttgttctaactggtactggtgagatctatttcaaaaccgcgcattttgaataa

SEQ ID NO: 8 - protein, 541 aa, Bacillus paralicheniformis
MKKRLSFISLMLIFTLVLSACGFGSSSGDGGKKDSKGKDTLNVNIKTEPFSLHPGLANDSVSANVLRQTFEGLTTIGKDGKPVEAAAEKIEVSDDQKTYTFTLRDAKWSNGDPVTAEDFEYAWKWALDPKNESQYAYQLYYLKGGEAANTGKGKIEDVGVKAVNDKTLKVELEKPTPYFTELTAFYTYMPVNKKVAEKNAKWYTNADENYVSNGPFKMAKWKHSGNIVLEKNDQYWDKDAVKLKKINMAMVNDPNTGLNMYKKGELDFVGQPLDQISTDAIPSLKKEGLNIDPFASVYLYKFNTEAAPLNNVNIRKALTYAINREAIVKNITQAEQLPAMGLVPPAVHGFESNKGYFKDHDVDKAKEYLEKGLKELGLKKASDLPKITLSFNTDEAHQKIAQAVQEMWNKELGLDVELGNEEWNVYIDKLHAGNYQIGRLGWTADFNDGMNFLETYRDKEGGNNDTNWENAKYKELLNKASRETDSAKRIELMKEAESIIMDELPVAPIYFYTMPYLHDESLKDFVLTGTGEIYFKTAHFE

(19) United States
(12) Patent Application Publication
(10) Pub. No.: US 2026/0271940 A1
(43) Pub. Date: Sep. 17, 2026

(54) COMPOSITION COMPRISING BIOFILM FORMING BACILLUS

(71) Applicant: CHR. HANSEN A/S, Hoersholm (DK)

(72) Inventors: Patricia Dominguez CUEVAS, Hoersholm (DK); Rute NEVES, Hoersholm (DK); Raquel AZEVEDO, Hoersholm (DK); Lars MOELBAK, Hoersholm (DK); Lorenzo FIMOGNARI, Hoersholm (DK)

(73) Assignee: CHR HANSEN A/S, Hoersholm (DK)

(21) Appl. No.: 19/366,180

(22) Filed: Oct. 22, 2025

Related U.S. Application Data
(63) Continuation of application No. 17/613,902, filed on May 28, 2020, now Pat. No. 12,478,069.

(30) Foreign Application Priority Data
May 29, 2019 (EP) 19177378.7

(51) Int. Cl. A01N 63/22; C12N 1/205; C12N 1/36; C12R 1/07

(52) U.S. Cl. CPC A01N 63/22; C12N 1/205; C12N 1/36; C12R 2001/07

(57) ABSTRACT

Present invention relates to new strains of Bacillus paralicheniformis with improved ability to form biofilm and colonize the rhizosphere when compared to their parental strains from where they are derived.

### CROSS-REFERENCE TO RELATED APPLICATIONS

This application is a continuation of Ser. No. 17/613,902, filed on Nov. 23, 2021, which is a national stage entry PCT/EP2020/064892, filed May 28, 2020, which claims priority to EP19177378.7, filed May 29, 2019. The contents of the aforementioned applications are fully incorporated herein by reference.

### SEQUENCE LISTING

This application contains a Sequence Listing in computer readable form, which is hereby incorporated by reference. The name of the file containing the Sequence Listing is SQ.xml, which was created on Oct. 16, 2025, and which contains 15,799 bytes.

### SUMMARY OF THE INVENTION

The present invention relates to a composition comprising Bacilli, Bacillus paralicheniformis and Bacillus licheniformis with improved biofilm forming properties on plants and/or its habitat, to its use, to a process for its preparation, to the use of Bacilli, Bacillus paralicheniformis and Bacillus licheniformis with improved biofilm forming properties for controlling, combating and/or conferring specific resistance to plant pests. Particularly, the invention relates to strains of Bacilli not expressing functional proteins encoded by one or more of the genes GntR, OppA, PdeH.

The inventors of present invention have selected derivative strains that show an increase in pellicle biofilm formation and differential colony structure. The derivative strains were selected after carrying out an adaptive laboratory evolution campaign. Genotypic variations associated with the phenotypic changes were detected and derivative strains have been tested in plant experimental systems and proven to promote increased plant growth as compared to the original (parental) strain.

Present disclosure explains how the genetic modifications have altered biosynthesis levels of biofilm components and how this promotes bioactivity of bioprotective Bacillus strains.

### FIELD OF THE INVENTION

In the current context of a modern and ecologic society, which is concerned with preserving the environment, biological control is considered an attractive alternative or supplement to conventional methods of control. Biological control is the use of one organism (predator, parasite or pathogen) that attacks another organism which is causing economic damage to crops. This is a very common strategy in agro ecological systems, as well as in conventional agriculture which relies on the Integrated Pest Management (IPM).

Although biological control brings positive effects in the reduction or withdrawal of pesticide use and improving farmers' income, an analysis of the set of experiments worldwide shows that the results are still concentrated in only a few crops. There is still much to develop in areas of control of pests and diseases.

There has been great emphasis on research on biological control with the use of bacteria colonizing the roots of plants, called rhizobacteria. The beneficial rhizobacteria for promoting growth and/or acting in the biological control of plant pathogenic bacteria are called plant growth-promoting rhizobacteria or PGPR.

One of the key factors for successful biological control by PGPR is successful colonization of the habitat e.g. by biofilm formation. Hence successful biofilm formation may increase the protective effect of the PGPR.

### STATE OF THE ART

The pressure of society to replace chemicals with environmentally acceptable products or ecological techniques has encouraged the search for alternative methods to promote plant health. In this context, biological control has been considered one of the alternatives within an integrated approach, in which one seeks to ensure sustainable development of agriculture.

The risks to humans and environments presented by using synthetic pesticides emphasize the need for tools such as biological control in optimizing sustainable agricultural systems.

Based on the idea that improved biofilm formation may improve the bioprotective effect of Bacilli, the inventors of present invention have selected derivative strains of Bacilli that show an increase in pellicle biofilm formation and differential colony structure.

To the best of our knowledge specific genetic features linked to improved biofilm formation and associated mode of actions have never been described for Bacilli and in particular not for B. paralicheniformis strains.

The function of the gene PdeH as c-di-GMP phosphodiesterase controlling c-di-GMP intracellular levels has been described previously in laboratory strains of B. subtilis, B. amyloliquefaciens and B. cereus by e.g. Kampf J, Stülke J. (2017). No studies have been done in B. licheniformis or B. paralicheniformis strains.

The functional role of GntR regulator is unknown. Therefore, investigating the molecular mechanism by which it generates more robust biofilms will constitute an undescribed mode of action and a contribution to the scientific field.

### BRIEF DESCRIPTION OF THE DRAWINGS

FIG. 1—ALE experimental approach to select for improved biofilm formers was followed for 12 weeks. Every 3-5 days, pellicles formed were collected with a sterile inoculation loop into a cryotube. Pellicles were disrupted, and aliquots were used to inoculate fresh medium in a new multi-well plate. At weeks 4, 7 and 12, pellicles were disrupted, and serial dilutions were plated on LB agar plates for independent clone selection. Individual clones were compared in biofilm formation with parental strain. Best biofilm formers were selected for further evolution steps or as final lineages. Temperature was gradually decreased from 25° C. to 18° C. throughout the ALE progression.

FIG. 2—Selected improved biofilm derivatives and biofilm quantification. FIG. 2A. Top view image of the pellicle biofilm formation experiment comparing the parental strains B. paralicheniformis DSM33110 with first 7 biofilm-improved strains iPH2970 M1-M7 (DSM33111-DSM33117, respectively). FIGS. 2B-2D. Summary of biofilm quantification results for DSM33110 and biofilm improved derivatives iPH2970 M2-M6. FIG. 2B. Corresponds to measured absorbance values at 570 nm obtained after crystal violet biofilm staining. Error bars indicate standard deviation between duplicates. FIG. 2C. Corresponds to measured absorbance values at 504 nm obtained after 5 and 6 h pellicles incubation with a 0.02% TTC solution. Error bars indicate standard deviation between duplicates. FIG. 2D. Corresponds to number of CFUs per pellicle re-suspended in 1 ml of MSgg medium. Error bars indicate standard deviation between duplicates.

FIG. 3—Colony morphology phenotypes compared between parental strain DSM33110 and biofilm-improved derivatives DSM33111-DSM33117. Images correspond to top view images of colony structures formed by the parental B. paralicheniformis DSM33110 and biofilm improved derivatives (iPH2970 M1-M7, DSM33111-DSM33117). 10 μl of bacterial pre-cultures (OD₆₀₀ 1, LB broth) were inoculated in the center of Petri dishes containing MSgg solid medium, supplemented or not with congo red dye (20 μg/ml Congo Red, CR). Plates were incubated at 30° C. for 3 days before images were collected.

FIG. 4—Swarming diameters reached by colonies of DSM33110 (or parental) and biofilm improved derivatives (iPH2970 M1-M7). Swarming halo diameters measured in MSgg medium at different temperatures (25-30-37° C.). Error bars correspond to standard deviation between 4 replicates (2×2 biological replicates).

FIG. 5—Plant growth and fitness promotion quantification results from DSM31100 (or parental) and 7 biofilm improved derivative strains (M1-M7, corresponding to DSM33111-DSM33117 respectively). FIG. 5A. Corresponds to the total leaf area measurements average calculated per plant (48 plants per strain). FIG. 5B. Corresponds to the average fresh weight per plant. FIG. 5C. Corresponds to chlorophyll quantification results normalized by fresh weight. Error bars correspond to standard deviation between samples.

FIG. 6—RT-qPCR expression analysis of biofilm related genes (epsA, epsE, tasA, pgsB and slr). Fold changes (relative expression levels) between DSM33110 and biofilm improved derivatives DSM33112-DSM33113-DSM33115 and DSM33116 (iPH2970 M2-M3-M5-M6, respectively) are represented in the plots. FIG. 6A. Fold change (relative expression) compared between bacterial cultures growing in planktonic growth conditions. FIG. 6B. Relative fold change expression levels compared between bacterial cultures growing in biofilm growth conditions. Fold change expression was normalized to expression in the parental strain DSM33110.

FIG. 7—RT-qPCR expression analysis of GntR regulated genes (azlC and brnQ), the global regulator CodY and its regulated gene ybgE. Fold change (relative expression levels) between DSM33110 and biofilm improved derivatives DSM33112-DSM33113-DSM33115 and DSM33116 (iPH2970 M2-M3-M5-M6, respectively) is represented in the plots. Fold change (relative expression) compared between bacterial cultures growing in planktonic growth conditions. Fold change expression was normalized to expression in the parental strain DSM33110.

FIG. 8—γ-PGA purification, gel electrophoresis and quantification. FIG. 8A. Scanned image corresponding to SDS-PAGE gel stained with methylene blue. γ-PGA samples correspond to biofilm culture supernatants of strains DSM33110 and derivatives DSM33112-DSM33113-DSM33115 and DSM33116 (iPH2970 M2-M3-M5-M6, respectively). FIG. 8B. Scanned image corresponding to SDS-PAGE gel stained with methylene blue. γ-PGA samples correspond to planktonic culture supernatants FIG. 8C. Plots corresponds to results from band intensity quantification from gels shown in A/B. Results are expressed as relative levels in respect to the parental strain DSM33110 (Parental strain), assigning a value of 100% to the γ-PGA produced by the original strain in each of the two growth conditions.

FIG. 9—Plant growth promotion by biofilm improved derivatives compared in A. thaliana grown in potting soil. FIG. 9A. Plant growth and fitness promotion quantification results from parental strain (DSM33110) and 7 biofilm improved derivative strains (M1-M7, corresponding to DSM33111-DSM33117 respectively). A. thaliana seedlings pre-grown in potting soil for 7 days, were inoculated by root dipping on bacterial cultures resuspended in 10 mM MgSO₄ buffer (OD₆₀₀ 0.1). Plantlets were allowed to grow for 14 additional days before data collection. FIG. 9B. Plant growth and fitness promotion quantification results from parental strain (DSM33110) and a formulated product combining biofilm improved derivative M3 (DSM33113) with B. subtilis strain and DSM32324. A. thaliana seedlings pre-grown in potting soil for 7 days were inoculated by root dipping on bacterial cultures resuspended in 10 mM MgSO₄ buffer (OD₆₀₀ 0.01). Plantlets were allowed to grow for 14 additional days before data collection. Error bars correspond to standard deviation between replicates and statistical significance was determined by performing a t-test (p<0.05) assuming equal variance in the two samples.

FIG. 10—Plant growth promotion by strain combination of biofilm improved derivative DSM33113 (M3) and B. subtilis strain DSM32324. Maize plant growth promotion quantification results for strain combination containing biofilm improved derivative DSM33113 (M3) with B. subtilis strain DSM32324. Maize seedlings pre-grown for 14 days were inoculated by root dipping on bacterial cultures resuspended in 10 mM MgSO₄ buffer (OD₆₀₀ 0.1). Plants were grown 14 days post-inoculation before data collection. FIG. 10A. Shoot fresh weight results from maize plants grown in clay soil (left) or sandy soil (right). FIG. 10B. Shoot dry weight results from maize plants grown in clay soil (left) or sandy soil (right). Error bars correspond to standard deviation between replicates.

### DETAILED DESCRIPTION OF THE INVENTION

### Rhizobacteria

The soils are home to a complex biological community, of which micro-organisms, prokaryotes and eukaryotes form a majority, both in number and in diversity. Some prokaryotes have ecological niches as the rhizosphere, and/or the rhizoplane of plants, where they multiply, survive and protect themselves from the rest of the antagonistic action of soil microflora. These organisms have been generically called rhizobacteria.

In association with plants, rhizobacteria may have a deleterious effect, null or beneficial. Those who exercise a beneficial effect—growth promotion and biological control of disease—are called PGPR (“Plant Growth-Promoting Rhizobacteria). It is estimated that only 1% to 2% of rhizobacteria have some beneficial effect for the plant with which they are associated.

### PGPR as Biocontrol Agents

PGPR have been used for biological control of plant diseases and thereby increase the productivity of crops. How and why this biological control is exercised is still a topic that needs complementary studies.

In some situations, it is possible that biological control occurs by direct antagonism exerted by PGPR against the pathogen, with involvement of the known mechanisms of antibiosis: production of antimicrobial substances, direct parasitism, competition for nutrients and ecological niches. Research has shown that certain PGPR appear to act as elicitor of ISR (induced systemic resistance), in the sense that the plant becomes systemically protected against more than one pathogen, unlike the classical biological control, which aims to implement the control more specifically.

A significant parameter affecting the PGPR ability to infect and colonize the plant surface is the ability of the PGPR to form biofilm. The inventors of present disclosure therefore seek to improve the biofilm and the plant growth promoting capabilities the Bacilli disclosed herein.

### The Rhizosphere Environment

One of the most convenient methods of introducing rhizobacteria in the root environment is through the application on the seeds before sowing. The process of seed germination releases carbohydrates and amino acids in abundance in the form of seed exudates. Thus, these organisms introduced with the seeds in the soil utilize exudates as a source of nutrition and colonize the roots as they emerge. Rhizobacteria isolates that have greater ability to utilize root exudates of seeds may have selective advantage in colonization of roots.

PGPR of the genus Bacillus have been associated with nematode control. Sikora, R. A. (Interrelationship between plant health promoting rhizobacteria, plant parasitic nematodes and soil microorganisms. Medicine Faculty Landbouww Rijksuniv Gent, Landbouww, v. 53, n.2b, p. 867-878, 1988) observed reductions in infection of Meloidogyne arenaria, M. incognita and Rotylenchulus reniformis around 60-65% with treatment of seeds of various crops with a strain of Bacillus.

### Advantages of Rhizobacteria for Commercial Application

The rhizobacteria have a number of advantages over chemical pesticides or even on other biological control agents: they are easy to mass-produce; they are easy to store and are adaptable to formulation technology.

The rhizobacteria can be applied by treating the substrate, immersing the seedling root systems in bacterial suspensions, watering the plant with bacterial suspension by dipping/coating the seeds in suspension of rhizobacteria or by applying PGPR with the pelleting of seeds.

### Bacteria of the Genus Bacillus

The Bacillus species are Gram-positive bacteria characterized by having thick cell walls and the absence of outer membranes, which differs from the Gram-negative bacteria. Much of the cell wall of Gram-positive bacteria is composed of peptidoglycan.

Gram-positive species are divided into groups according to their morphological and biochemical characteristics. The genus Bacillus belongs to the group of sporulating bacteria. Bacterial spores are one of the most resilient cell types; they resist any environmental changes, withstand dry heat and certain chemical disinfectants and may persist for years on dry land.

The beneficial effect of Bacilli such as e.g. B. paralicheniformis, when applied near the seed or the soil, is not solely due to the antagonism afforded to pathogens. The PGPR has a positive influence on germination, development and crop yield due also to the production of substances which promote growth and improvement in plant nutrition by solubilization of phosphorus.

Current activities aim at showing the differential regulation in expression of biofilm components biosynthetic pathways, including EPS, TasA amyloid-like protein and γ-PGA, between the wt and the derivative biofilm-improved strains iPH2970 M2, M3, M5 and M6 (DSM33112, DSM33113, DSM33115 and DSM33116, respectively). In addition, genetic complementation studies will allow linking each SNP with specific phenotypic changes, namely pellicle and colony structure changes, as well as swarming motility differences.

Thus, the first aspect of the invention relates to the herein described novel strains or mutants thereof.

The composition of the present invention may, besides the active components, contain agrochemical acceptable excipients and/or vehicles thereof. The composition of the invention further comprises agrochemically acceptable carriers, vehicles and/or adjuvants.

Among the main crops of plants are sugar cane, coffee, soybeans, cotton, corn, potatoes, tomatoes, tobacco, banana, rice, wheat, avocado, pineapple, squash, cacao, coconut, oats, onion, lettuce, beet, carrot, cassava, beans, sunflower, pepper, turnip, apple, strawberry, okra, radish and onion.

With regard to fruticulture: citrus, grape, guava, papaya, fig, peach, plum and nespereira or loquat are of particular relevance and with regard to horticulture: eggplant and cruciferous.

With regard to floriculture: rose, chrysanthemum, lisianthus, gerbera, amaryllis, begonia and celosia.

The composition of present invention may be coated on the plant seed and can include an amount of Bacillus, such as e.g. B. paralicheniformis spores from about 1.0×10² CFU/seed to about 1.0×10⁹ CFU/seed.

The plant seed can include, but is not limited to, the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

In one or more embodiments, the plant seed can include seed of a drybean, a corn, a wheat, a soybean, a canola, a rice, a cucumber, a pepper, a tomato, a squash, a cotton, a grass, and a turf grass.

In an alternative embodiment, the Bacillus or composition of present invention may be added to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one or more embodiments, the plant can include soybean, bean, snap bean, wheat, cotton, corn, pepper, tomato, potato, cassava, grape, strawberry, banana, peanut, squash, pumpkin, eggplant, and cucumber.

In the compositions and methods of the present invention, the pathogenic infection can be caused by a wide variety of plant pathogens including, for example, but not limited to, a plant fungal pathogen, a plant bacterial pathogen, a rust fungus, a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium culmorum, a Fusarium graminearum, a Fusarium oxysporum, a Fusarium oxysporum f. sp. Cubense, a Fusarium oxysporum f. sp. Lycopersici, a Fusarium virguliforme, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae, an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

In some embodiments, the pathogenic infection can be caused by one or a combination of: Soybean rust fungi (Phakopsora pachyrhizi, Phakopsora meibomiae) and the plant comprises soybean; Botrytis cinerea (Botrytis Blight) and the plant comprises grape; Botrytis cinerea (Botrytis Blight) and the plant comprises strawberry; Botrytis cinerea (Botrytis Blight) and the plant comprises tomato; Alternaria spp. (e.g. A. solani) and the plant comprises tomato; Alternaria spp. (e.g. A. solani) and the plant comprises potato; Bean Rust (Uromyces appendiculatus) and the plant comprises common bean; Microsphaera diffusa (Soybean Powdery Mildew) and the plant comprises soybean; Mycosphaerella fijiensis (Black sigatoga) or Fusarium oxysporum f. sp. cubense (Panama disease) and the plant comprises banana; Xanthomonas spp, or Xanthomonas oryzae pv. oryzae and the plant comprises rice; Xanthomonas axonopodis and the plant comprises cassava; Xanthomonas campestris and the plant comprises tomato; Botrytis cinerea (Pepper Botrytis Blight) and the plant comprises pepper; Powdery mildew and the plant comprises a cucurbit; Sclerotinia sclerotiorum (white mold) and the plant comprises snap bean; Sclerotinia sclerotiorum (white mold) and the plant comprises potato; Sclerotinia homeocarpa (dollar spot) and the plant comprises turfgrass; Southern White Mold and the plant comprises peanut; Leaf spot (Cercospora/Cercosporidium) and the plant comprises peanut; Fusarium graminearum (Wheat Head Scab) and the plant comprises wheat; Mycosphaerella graminicola (Septoria tritici blotch) and the plant comprises wheat; Stagonospora nodorum (glume blotch and Septoria nodorum blotch), and the plant compromises wheat; Erwinia amylovora, and the plant compromises apple, pear and other pome fruits; Venturia inaequalis, and the plant compromises apple, pear and other pome fruits; or Rhizoctonia solani and the plant comprises wheat, rice, turfgrass, soybean, corn, legumes and vegetable crops. The compositions including the bacilli as described herein strain can be in the form of a liquid, an oil dispersion, a dust, a dry wettable powder, a spreadable granule, or a dry wettable granule. More specifically the composition may for example be an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

The present invention relates to a composition comprising Bacillus paralicheniformis DSM33111, DSM33112, DSM33113, DSM33114, DSM33115, DSM33116 or DSM33117, or mutants thereof or a mutant thereof, and to a kit comprising the composition, or prepared by the process of preparing the composition, as well as instructions and a suitable recipient.

A process for preparing a composition comprising Bacillus paralicheniformis DSM33111, DSM33112, DSM33113, DSM33114, DSM33115, DSM33116 or DSM33117, or a mutant thereof together with agrochemically acceptable carriers, vehicles and/or adjuvants, and use of said composition for controlling, combating and/or conferring specific resistance to phytonematodes are also given.

In addition, the invention refers to the use of effective amounts of Bacillus paralicheniformis DSM33111, DSM33112, DSM33113, DSM33114, DSM33115, DSM33116 or DSM33117 mutant thereof, in the manufacture of an agrochemical composition with plant growth promoting effect in a plant culture, as well as processes for promoting plant health.

In the context of the present invention, original strain or parental strain or mother strain or wt strain or parental B. paralicheniformis or DSM 33110 are synonyms and are herein interchangeable.

In the context of the present invention, M1 or DSM 33111 or Bacillus paralicheniformis DSM 33111 are synonyms and are herein interchangeable.

In the context of the present invention, M2 or DSM 33112 or Bacillus paralicheniformis DSM 33112 are synonyms and are herein interchangeable.

In the context of the present invention, M3 or DSM 33113 or Bacillus paralicheniformis DSM 33113 are synonyms and are herein interchangeable.

In the context of the present invention, M4 or DSM 33114 or Bacillus paralicheniformis DSM 33114 are synonyms and are herein interchangeable.

In the context of the present invention, M5 or DSM 33115 or Bacillus paralicheniformis DSM 3315 are synonyms and are herein interchangeable.

In the context of the present invention, M6 or DSM 33116 or Bacillus paralicheniformis DSM 33116 are synonyms and are herein interchangeable.

In the context of the present invention, M7 or DSM 33117 or Bacillus paralicheniformis DSM 33117 are synonyms and are herein interchangeable.

In the context of the present invention, mock or control or non-inoculated plants are synonyms and are herein interchangeable.

In the context of the present invention, a mutation is to be understood as an alteration in the wild-type nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift. In the context of the present invention, a deletion is to be understood as a genetic mutation resulting in the removal of one or two nucleotides of wild-type nucleotide sequence of the genome of an organism; a insertion is to be understood as the addition of one or more nucleotides to the wild-type nucleotide sequence; a substitution (or point mutation) is to be understood as a genetic mutation where a nucleotide of wild-type nucleotide sequence is changed by another nucleotide; a frameshift is to be understood as a genetic mutation caused by a insertion or deletion of a number of nucleotides in a wild-type nucleotide sequence that is not divisible by three, therefore changing the reading frame and resulting in a completely different translation from the original reading frame; an introduction of a stop codon is to be understood as a point mutation in the DNA sequence resulting in a premature stop codon; a inhibition of substrate binding of the encoded protein is to be understood as any mutation in the nucleotide sequence that leads to a change in the protein sequence responsible for preventing binding of a substrate to its catalytic site of the protein. Furthermore, a knockout mutant is to be understood as genetic mutation resulting in the removal or deletion of a gene, such as an entire gene or an entire open reading frame from the genome of an organism.

Algorithms for aligning sequences and determining the degree of sequence identity between them are well known in the art. For the purpose of the present invention and as an example, one of these algorithms is based on aligning both sequences with the blastp as provided by the National Center for Biotechnology Information (NCBI) at the blast.ncbi.nlm.nih.gov website applying standard parameter settings (Matrix: BLOSUM62, Gap Costs: Existence: 11 Extension: 1, Conditional compositional score matrix adjustment) and subsequent quantification of identical amino acid pairs in identical positions over the aligned amino acid sequences. A similar process may be carried out for aligning nucleotide sequences using, in this case, blastn as provided by the National Center for Biotechnology Information (NCBI) at the blast.ncbi.nlm.nih.gov website applying standard parameter.

The following listed aspects are further comprised by present invention:

Aspect 1. A Bacillus having a mutation in the pdeHI, oppA and/or gntR gene, such as a Bacillus having a mutation in the pdeH gene, oppA gene, gntR gene, pdeHI and oppA genes, pdeH and gntR genes, oppA and gntR genes, pdeH and oppA and gntR genes, preferably a Bacillus having a mutation in the pdeH gene, gntR gene, oppA and gntR genes, pdeH and oppA and gntR genes.

Aspect 2. A Bacillus having a mutation in the pdeH, oppA and/or gntR gene when compared to the corresponding ortholog genes in B. paralicheniformis deposited as DSM33110, such as a Bacillus having a mutation in the pdeH gene, oppA gene, gntR gene, pdeH and oppA genes, pdeH and gntR genes, oppA and gntR genes, pdeHI and oppA and gntR genes, preferably a Bacillus having a mutation in the pdeH gene, gntR gene, oppA and gntR genes, pdeH and oppA and gntR genes.

Aspect 3. A Bacillus according to any of the preceding aspects, wherein the closest ortholog of the pdeH gene of B. paralicheniformis deposited as DSM33110 share less than 100% such as e.g. less than 99%, less than 98%, less than 97% sequence identity with SEQ ID NO: 1.

Aspect 4. A Bacillus according to any of the preceding aspects, wherein the closest ortholog of the pdeH gene of B. paralicheniformis deposited as DSM33110 share at least 95% such as e.g. at least 96%, at least 97%, at least 98%, at least 99% sequence identity with SEQ ID NO: 1.

Aspect 5. A Bacillus according to any of the preceding aspects, wherein the closest ortholog of the oppA gene of B. paralicheniformis deposited as DSM33110 share less than 100% such as e.g. less than 99%, less than 98%, less than 97% sequence identity with SEQ ID NO: 3 or 7.

Aspect 6. A Bacillus according to any of the preceding aspects, wherein the closest ortholog of the oppA gene of B. paralicheniformis deposited as DSM33110 share at least 95% such as e.g. at least 96%, at least 97%, at least 98%, at least 99% sequence identity with SEQ ID NO: 3 or 7.

Aspect 7. A Bacillus according to any of the preceding aspects, wherein the closest ortholog of the gntR gene of B. paralicheniformis deposited as DSM33110 share less than 100% such as e.g. less than 99%, less than 98%, less than 97% sequence identity with SEQ ID NO: 5.

Aspect 8. A Bacillus according to any of the preceding aspects, wherein the closest ortholog of the gntR gene of B. paralicheniformis deposited as DSM33110 share at least 95% such as e.g. at least 96%, at least 97%, at least 98%, at least 99% sequence identity with SEQ ID NO: 5.

Aspect 9. A Bacillus according to any of the preceding aspects, wherein the mutation is a deletion, substitution or insertion, preferably the mutation in the oppA gene causes a deletion of 2 codons coding for threonine and tyrosine in positions 98 and 99, respectively, of the OppA protein and/or preferably wherein the mutation in the gntR gene causes a substitution in position 311 of GntR protein, such as substitution in position Lys311 of the GntR protein, such as Lys311Glu.

Aspect 10. A Bacillus according to any of the preceding aspects, wherein the mutation causes a frameshift, introduces a stop codon or inhibits substrate binding of the encoded protein, preferably wherein the mutation in the pdeH gene causes a frameshift or introduces a stop codon, more preferably wherein the mutation in the pdeH gene introduces a stop codon.

Aspect 11. A Bacillus according to any of the preceding aspects, wherein the protein encoded by one or more of the genes pdeHI, oppA and/or gntR or one or more of their closest orthologs is rendered dysfunctional by the mutation.

Aspect 12. A Bacillus according to any of the preceding aspects, wherein the genome of the strain is at least 99%, such as e.g. at least 99.5%, such as e.g. at least 99.8%, such as e.g. at least 99.9% identical to the genome of the strain deposited at Deutsche Sammlung von Mikroorganismen und Zellkulturen with accession No. DSM33110.

Aspect 13. A Bacillus according to any of the preceding aspects, wherein the Bacillus is selected from: Bacillus licheniformis, Bacillus amyloliquefaciens, Bacillus paralicheniformis, Bacillus cereus, Bacillus velezensis, Bacillus megaterium, preferably Bacillus paralicheniformis.

Aspect 14. A Bacillus according to any of the preceding aspects, wherein the strain is derived from the strain deposited at Deutsche Sammlung von Mikroorganismen und Zellkulturen with accession No. DSM33110 or a strain sharing phenotypical characteristics with the strain deposited at Deutsche Sammlung von Mikroorganismen und Zellkulturen with accession No. DSM33110.

Aspect 15. A Bacillus according to any of the preceding aspects wherein the Bacillus has the phenotypical characteristics of one or more of the strains deposited at Deutsche Sammlung von Mikroorganismen und Zellkulturen with accession No's. DSM33111, DSM33112, DSM33113, DSM33114, DSM33115, DSM33116, DSM33117.

Aspect 16. A Bacillus paralicheniformis according to any of the preceding aspects wherein the B. paralicheniformis is selected from a list consisting of the strains deposited at Deutsche Sammlung von Mikroorganismen und Zellkulturen with accession No's. DSM33111, DSM33112, DSM33113, DSM33114, DSM33115, DSM33116, DSM33117.

Aspect 17. A Bacillus paralicheniformis according to any of the preceding aspects showing increased pellicle biofilm formation when compared to its parental strain deposited at Deutsche Sammlung von Mikroorganismen und Zellkulturen with accession DSM33110.

Aspect 18. A composition comprising a Bacillus according to any of the preceding aspects.

Aspect 19. A composition comprising a Bacillus according to any of the preceding aspects and agrochemically acceptable excipients and/or carriers thereof.

Aspect 20. The composition of any of aspects 18 or 19, further comprising one or a combination of a microbial, a biological, or a chemical insecticide, fungicide, nematicide, bactericide, herbicide, plant extract, plant growth regulator, or fertilizer present in an amount suitable to benefit plant growth and/or to confer protection against a pathogenic infection in a susceptible plant, a carrier, a surfactant, a dispersant, or a yeast extract, preferably wherein the microbial is a Bacillus strain or wherein the microbial is a Bacillus subtilis strain or wherein the Bacillus subtilis strain is a strain deposited at Deutsche Sammlung von Mikroorganismen und Zellkulturen with accession No DSM32324, more preferably wherein the composition of any of aspects 18 or 9 is a composition comprising B. paralicheniformis strain deposited at Deutsche Sammlung von Mikroorganismen und Zellkulturen with accession No. DSM33113 further comprising a Bacillus strain or a Bacillus subtilis strain or a Bacillus subtilis strain deposited at Deutsche Sammlung von Mikroorganismen und Zellkulturen with accession No DSM32324, even more preferably wherein the composition of any of aspects 18 or 9 is a composition comprising B. paralicheniformis strain deposited at Deutsche Sammlung von Mikroorganismen und Zellkulturen with accession No. DSM33113 and the Bacillus subtilis strain deposited at Deutsche Sammlung von Mikroorganismen und Zellkulturen with accession No DSM32324.

Aspect 21. Use of a composition according to any of aspects 18 to 20 or a Bacillus according to any of aspects 1 to 17 as a biostimulant and/or bionematocide and/or plant growth enhancer and/or plant health promotor and/or plant disease or pest controller.

Aspect 22. Use of a composition, according to any of aspects 18 to 21, or a Bacillus according to any of aspects 1 to 17 for controlling, combating and/or conferring specific resistance to phytonematodes.

Aspect 23. Use according to any of aspects 21 or 22, wherein the phytonematodes are selected from the group consisting of Meloidogyne, Pratylenchus, Heterodera, Globodera, Ditylenchus, Tylenchulus, Xiphinema, Radopholus, Rotylenchulus, Helicotylenchus and Belonolaimus.

Aspect 24. Use according to any of aspects 21 to 23, wherein the phytonematode is selected from the group consisting of Meloidogyne incognita, Meloidogyne javanica, Meloidogyne exigua, Meloidogyne paranaensis, Heterodera glycines and Pratylenchus zeae.

Aspect 25. Use according to any of aspects 21 to 24 wherein the composition according to any of aspects 18 to 20 or the Bacillus according to any of aspects 1 to 17 is applied on a plant, a seed or in the habitat of a plant.

Aspect 26. Use according to aspect 25 wherein the plant is selected from the group consisting of corn, rice, sugar cane, soybean, potato, carrot, coffee and banana.

Aspect 27. Process for conferring improved resistance to phytonematodes, comprising applying an effective amount of a Bacillus of any of aspects 1 to 17 or a composition according to any of aspects 18 to 20 on plants and/or their habitat.

Aspect 28. Kit, comprising the composition as defined in any one of aspects 18 to 20, instructions for use and a suitable container.

Aspect 29. A plant seed coated with a composition according to any of aspects 18 to 20 present in an amount suitable to benefit plant growth and/or to confer protection against a pathogenic infection in a susceptible plant.

Aspect 30. The plant seed of aspect 29, wherein the composition comprises an amount of Bacillus paralicheniformis according to any of aspects 1 to 17 spores from about 1.0×10² CFU/seed to about 1.0×10⁹ CFU/seed.

Aspect 31. The plant seed of aspect 30, wherein the composition further comprises one or a combination of a microbial, a biological, or a chemical insecticide, fungicide, nematicide, bacteriocide, or plant growth regulator present in an amount suitable to benefit plant growth and/or to confer protection against a pathogenic infection in a susceptible plant, preferably wherein the microbial is a Bacillus strain, more preferably wherein the microbial is a Bacillus subtilis strain, even more preferably wherein the Bacillus subtilis strain is a strain deposited at Deutsche Sammlung von Mikroorganismen und Zellkulturen with accession No DSM32324.

Aspect 32. A Bacillus according to any of the preceding aspects, wherein PdeH is encoded by SEQ ID NO: 1 or homologs thereof, OppA is encoded by SEQ ID NO: 3 or 7, preferably 7, or homologs thereof and/or GntR is encoded by SEQ ID NO: 5 or homologs thereof.

Aspect 33. A method of treating a plant to enhance plant growth and/or promote plant health and/or control a plant disease, wherein the method comprises the step of applying a Bacillus strain according to any of the preceding aspects 1 to 17 or the step of applying a composition according to any of the preceding aspects 18 to 20, wherein the said step enhances biofilm formation or enhances pellicle biofilm formation when compared to a method a) comprising a step of applying a Bacillus strain deposited at Deutsche Sammlung von Mikroorganismen und Zellkulturen with accession DSM33110 or b) comprising a step of applying a composition having Bacillus strain deposited at Deutsche Sammlung von Mikroorganismen und Zellkulturen with accession DSM33110.

Aspect 34. The method according to the preceding aspect, further comprising a step of applying a Bacillus strain according to any of the preceding aspects 1 to 17 or the composition according to any of the preceding aspects 18 to 20, to soil.

Aspect 35. The method according to any of the preceding aspects 33 to 34, wherein the step of applying a Bacillus strain according to any of the preceding aspects 1 to 17 or the composition according to any of the preceding aspects 18 to 20, is applied before, during or after the plant or plant part comes into contact with the soil.

Aspect 36. The method according to any of the preceding aspects 33 to 35, wherein the plant part is selected from the group consisting of a seed, root, corm, tuber, bulb and rhizome.

The illustrative examples presented below serve to better describe the present invention. However, the formulations described merely refer to some means to some embodiments of the present invention and should not be taken as limiting the scope thereof.

### EXAMPLES

### Example 1: Adaptive Laboratory Evolution (ALE)

In laboratory conditions, Bacillus biofilm formation can be assessed by comparing the robustness and structure complexity of floating pellicles in the liquid-air interface (pellicle biofilm). Pellicle formation was expected to act as a good indicator of the root colonization ability of a bacterial strain. It was therefore hypothesized that improving pellicle formation in a chosen strain could theoretically improve its properties as plant growth promoting bacteria (PGPB).

An experimental setup to select for better biofilm formers was therefore designed.

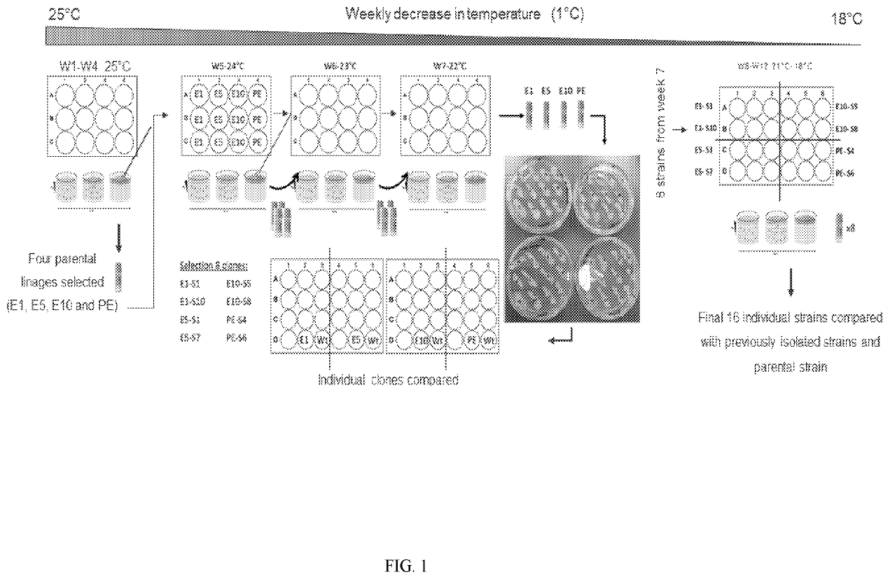

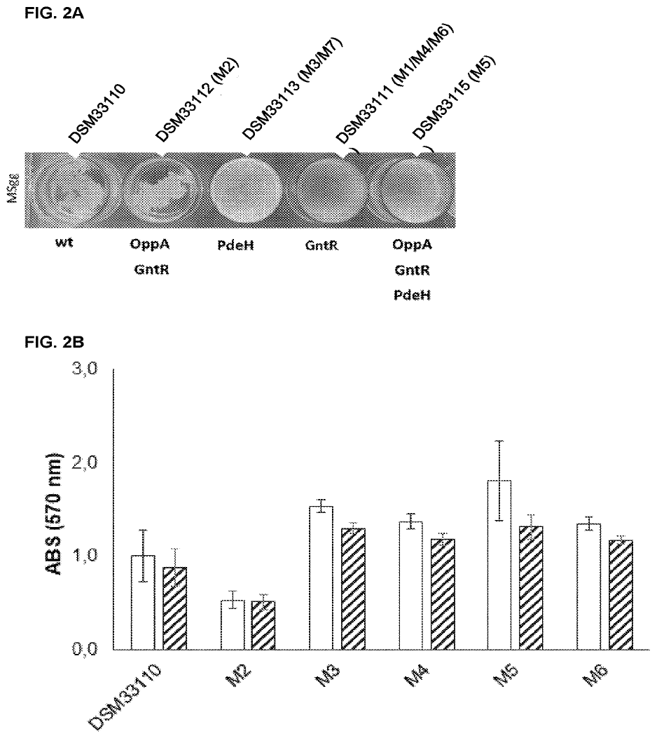

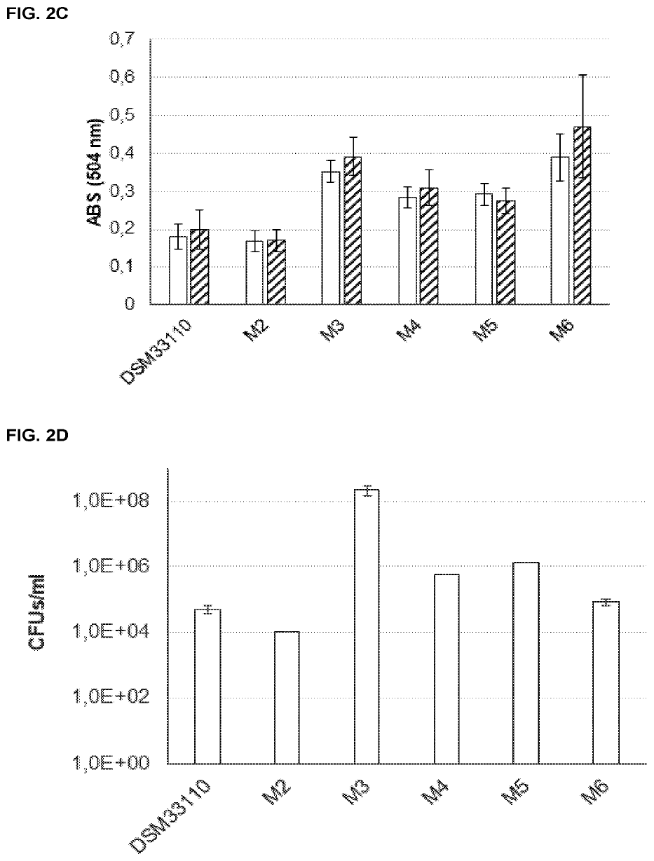

Chemically defined medium (MSgg) that promotes biofilm formation was inoculated with a stock of Bacillus paralicheniformis (DSM33110) cell culture (OD600 1). Multi-well plates were used throughout the 12 weeks span of experimental evolution (see FIG. 1).

During a span of 12 weeks experimental evolution, the Bacillus cells forming part of the pellicle were collected with a sterile inoculation loop after sufficient growth and transferred to a glycerol tube containing 1 ml of fresh MSgg medium. Pellicle biofilms were disaggregated by vortexing the tube/s thoroughly and pellicles were sheared into small pieces. 20 μl aliquots/well were used to inoculate a new multi-well plate for a new round of biofilm formation. During the first four weeks biofilm formation proceeded at 25° C., and afterwards temperature was decreased by 1° C. every week for 8 additional weeks, where temperature reached 18° C. as schematically depicted in FIG. 1.

Three independent selection steps were carried out, after weeks 4, 7 and 12. For each selection step, an aliquot from the re-suspended biofilm was plated on LB agar plates to obtain independent colonies. Individual clones were then re-streaked on LB agar plates and used as inoculation material in the following week. Four initial clones were selected in week 4, that were further evolved until week 8. Then two clones per initial selected strain were chosen and finally after week 12, two more strains were isolated from each of the 8 derivatives selected at week 8. In total the selection regime resulted into 28 derivatives isolated, being the last 8+16 clones the most relevant ones that showed a truly differential phenotype compared to the mother strain (DSM33110).

Final selected clones (16) were deposited internally and genome sequence analysis and single nucleotide polymorphism (SNP) analysis was performed by comparing the genome sequences of the selected biofilm improved strains to the parental B. paralicheniformis (DSM33110) genome sequence.

### Example 2: Genome Sequence Analysis

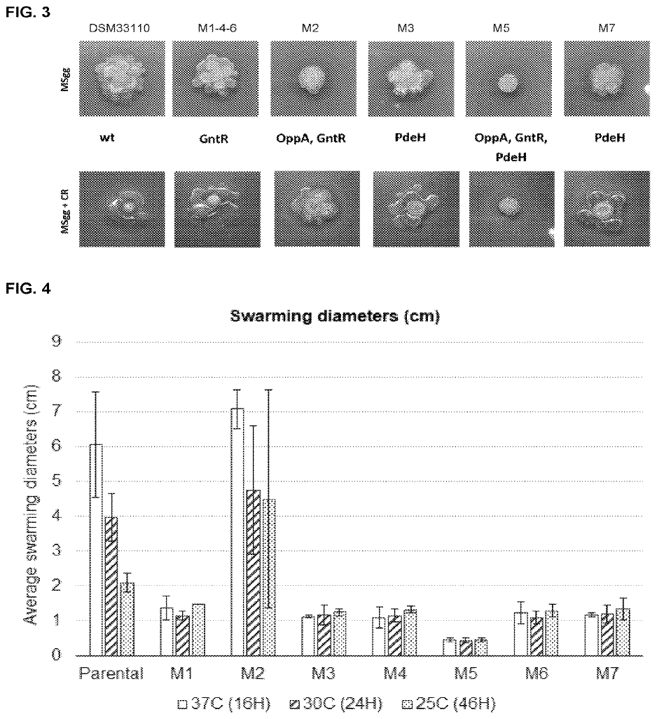

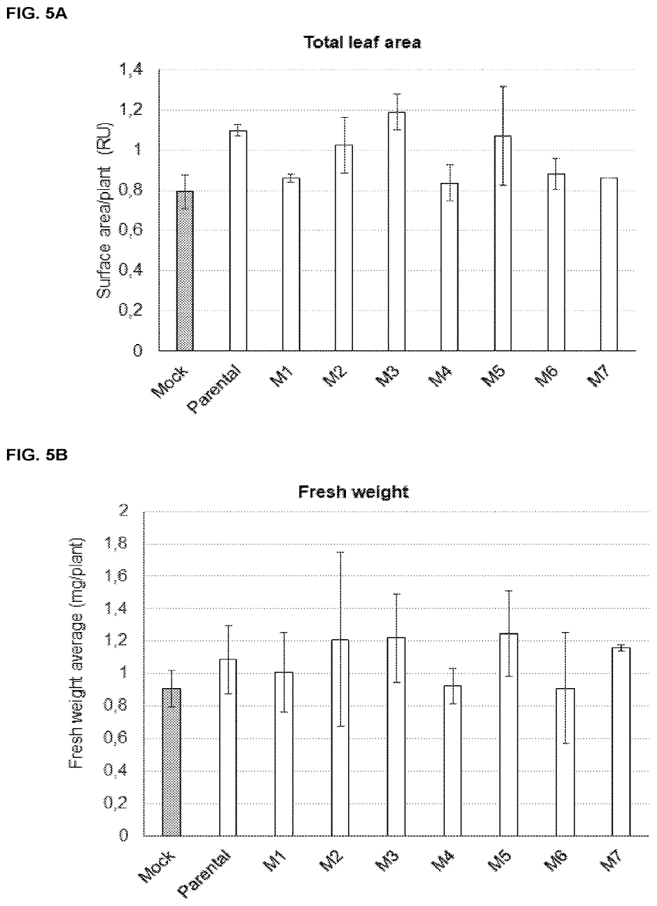

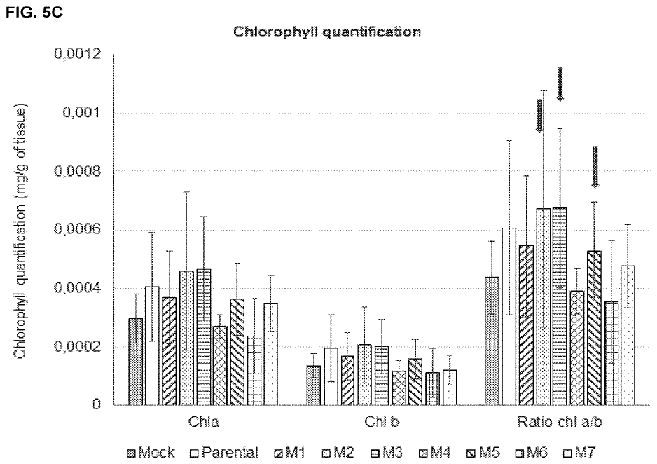

Out of 16 biofilm-improved derivatives sequenced, 8 showed mutations targeting the yuxH orf. yuxH is annotated as homologue of the c-di-GMP phosphodiesterase PdeH (yuxH) in B. subtilis strains (Chen, Y. et al. 2012). Cyclic di-GMP (c-di-GMP) is a second messenger that regulates diverse cellular processes in bacteria, including motility, biofilm formation, cell-cell signaling, and host colonization. Genetic studies have shown that c-di-GMP levels regulate swimming and swarming motility and influence biofilm formation, both in B. subtilis, B. cereus and B. amyloliquefaciens strains. All mutations found in yuxH were loss-of-function mutations because, either a stop codon or a frameshift in the coding sequence were introduced. Thus, selected strains with a mutation in yuxH most likely have an increased intracellular level of c-di-GMP which affects both motility and biofilm formation. We tested swarming motility in the derivative strains DSM33111 to DSM33117 and compared to the parental strain. Our results show a clear impairment in swarming motility for 6 out 7 mutants tested as compared to the mother strain (DSM33110). On the other hand, derivative strains bearing mutations in PdeH showed a clear increase in biofilm formation and colony structure phenotypes, in accordance with upregulated expression of the biofilm matrix components (see FIGS. 3-4).

TABLE 1
SNP analysis and ORF target identification. Table contains information linking
biofilm improved derivatives ID/name with the corresponding DSM number.
SNPs identity and frequency with which they are found are summarized.

| DSM | | | |
|---|---|---|---|
| number | Strain name | SNPs identified | Frequency |
| 33111 | IPH2970 M1 | Transcriptional regulator, GntR family domain/ | 12 out of 15 (80%) |
| | | Aspartate aminotransferase (EC2.6.1.1) Lys311Glu | |
| 33112 | iPH2970 M2 | Transcriptional regulator, GntR family domain/ | 12 out of 15 (80%) |
| | | Aspartate aminotransferase (EC2.6.1.1) Lys311Glu | |
| | | ABC transporter, periplasmic oligopeptide-binding | 2 out of 15 (13%) |
| | | protein OppA (TC 3.A.1.5.1) Deletion Thr98/Tyr99 | |
| 33113 | iPH2970 M3 | Alternative gene name: comB, yufA:p.Gln188* | 8 out of 15 (53%) |
| 33114 | iPH2970 M4 | Transcriptional regulator, GntR family domain/ | 12 out of 15 (80%) |
| | | Aspartate aminotransferase (EC2.6.1.1) Lys311Glu | |
| 33115 | iPH2970 M5 | Transcriptional regulator, GntR family domain/ | 12 out of 15 (80%) |
| | | Aspartate aminotransferase (EC2.6.1.1) Lys311Glu | |
| | | ABC transporter, periplasmic oligopeptide-binding | 2 out of 15 (13%) |
| | | protein OppA (TC 3.A.1.5.1) Deletion Thr98/Tyr99 | |
| | | Alternative gene name: comB, yufA:p.Arg7fs | 8 out of 15 (53%) |
| 33116 | iPH2970 M6 | Transcriptional regulator, GntR family domain/ | 12 out of 15 (80%) |
| | | Aspartate aminotransferase (EC2.6.1.1) Lys311Glu | |
| 33117 | iPH2970 M7 | Alternative gene name: comB, yufA:p.Gln188* | 8 out of 15 (53%) |

Single nucleotide polymorphism (SNP) analysis of genome sequences revealed the presence of a deletion in the orf oppA in two out of 15 improved biofilm strains. OppA is the extracellular binding protein of the Opp oligopeptide ABC transporter. OppA is an oligopeptide permease, involved in quorum sensing, sporulation and competence. OppA is also one of the major protein components of Bacillus floating biofilms together with polymers of glutamic acid (γ-PGA). The opp operon encodes an oligopeptide permease required for the import of the quorum sensing pentapeptide CSF, encoded by the phrC gene. CSF contributes to the activation of the ComA transcription factor, a response regulator required for competence development. In fact, the ComX-ComP-ComA signalling pathway constitutes a major quorum response pathway in B. subtilis, and it regulates the production of γ-PGA (Comella, N and Grossman, A. D, 2005; Stanley, N. R. and Lazazzera, B. A. 2005). It has also been reported that a defect in the oppA gene affects eDNA production. The opp operon is also induced upon cold stress or cold cultivation conditions in B. subtilis. Opp helps fine-tuning the levels of Spo0A, the master regulator during sporulation initiation, to ensure survival at low temperatures.

Genome sequencing results revealed the presence of a unique SNP in an orf annotated as GntR transcriptional regulator family. This GntR regulator belongs to the MocR family of DNA-binding transcriptional regulators, which contain an aminotransferase domain. A unique SNP was found in 12 out of 15 improved biofilm strains and that particular nucleotide change results in a single amino acid change of Lys311, involved in PLP co-enzyme binding, to Glu. Pyridoxal phosphate (PLP, pyridoxal 5′-phosphate, P5P) is the active form of vitamin B6. This amino acid change found in all 12 improved biofilm strains blocks PLP coenzyme binding. The genomic region around the GntR regulator is conserved in B. paralicheniformis species, but not in B. subtilis or B. amyloliquefaciens. We could speculate that the function of this protein is to regulate the expression of the adjacent azl operon, which encodes a branched-chain amino acid transporter. However, further experiments comparing global expression levels between derivatives and the parental strain are needed to understand the mode of action of this mutation in GntR.

### Example 3—Phenotypic Analysis on Agar Plates

Phenotypic changes in colony structure on agar plates revealed differences between the wt strain (DSM33110) and their derivatives. To better visualize changes in colony structure and matrix composition we decided to use MSgg solid media supplemented or not with 20 μg/ml Congo Red (CR) and 10 μg/ml Coomasie Brilliant Blue dyes to stain amyloid protein fibers (Romero et al. 2010). FIG. 3 shows top view images of colony structures generated by B. paralicheniformis (DSM33110) and evolved biofilm-improved strains DSM33111-DSM33117. Differences between the wt strain (DSM33110) and the biofilm improved derivatives could be easily appreciated even in the absence of Congo red. Derivative strains DSM33113, DSM33115 and DSM33117, bearing mutations in PdeH, show a higher degree of wrinkle formation in the center of the colony as compared with the mother strain (DSM33110). On the other hand, colonies from DSM33112 and DSM33115 appeared drier as compared to the wt strain or derivatives DSM33111 and DSM33113, where the deletion in oppA is absent. This difference in the mucoid, slimly aspect of the colonies could be attributed to differences in the amount of γ-polyglutamate being produced, which could be linked to the presence of a deletion in the oppA gene.

### Example 4—Swarming Motility

Swarming motility is defined as a rapid multicellular movement of bacteria across a surface, powered by rotating flagella and highly dependent on biosurfactants production. Biofilm forming, and swarming/swimming motile cells are oppositely regulated in Bacillus subtilis. In addition, it has been reported previously that mutations in yuxH (pdeH) decrease swarming motility. Therefore, we performed swarming motility assays to compare the parental strain (DSM33110) and biofilm improved derivatives.

Swarming diameters were measured for each strain in duplicates at three different temperatures (25° C., 30° C. and 37° C.). Experiments were performed twice, and results are the average of the 4 replicates per strain and condition. Data shown in FIG. 4.

### Example 5—Plant Growth Promotion Activity

Plant growth promotion efficiency was compared between the derivative strains using A. thaliana plants, in a soil system, and in a gnotobiotic system based on the use of 24-well plates filled with plant growth medium solidified with agar. Based on the results from three independent experiments, both in soil and agar systems, derivative strains iPH2970 M2, M3 and M5 were selected (DSM33112, DSM33113 and DSM33115, respectively) as the best biofilm-improved (DSM33110) derivatives.

### Example 6—Changes in Gene Expression

To evaluate changes in gene expression between DSM33110 and its derivative strains, both in planktonic and biofilm growth conditions, we have performed real-time qPCR analysis using specific primers for biofilm related genes. In parallel, biochemical analysis of extracellular matrix components was carried out. A method for purification and quantification of γ-PGA was implemented. Finally, we looked at the effect of the identified mutations in surface attachment experiments, and the improved performance of selected derivatives in plant growth experiments.

### Example 7—Analysis of Relative Gene Expression Levels of Biofilm Biosynthesis Related Genes (Real Time qPCR Gene Expression Quantification)

We collected samples from planktonic or biofilm growing cultures corresponding to the parental strain DSM33110 and biofilm improved derivatives carrying mutations in one, two or three of the previously described gene targets. We isolated total mRNA from both planktonic cells and biofilm of DSM33110 (Parental) and derivatives DSM33112 (M2), DSM33113 (M3), DSM33115 (M5), and DSM33116 (M6).

Next, we carried on reverse transcription (RT) followed by qPCR with specific primers for selected genes, either involved in biofilm biosynthesis regulation or biofilm matrix component biosynthesis. Differential gene expression between DSM33110 and biofilm improved derivatives was tested for genes related to biofilm matrix components biosynthesis or biofilm regulation (i.e. epsA, epsE, tasA, pgsB and slr). Expression of control genes (rpoB, strain specific primer pair DSM33110) was also analyzed by qPCR and used to normalize cDNA differences between samples.

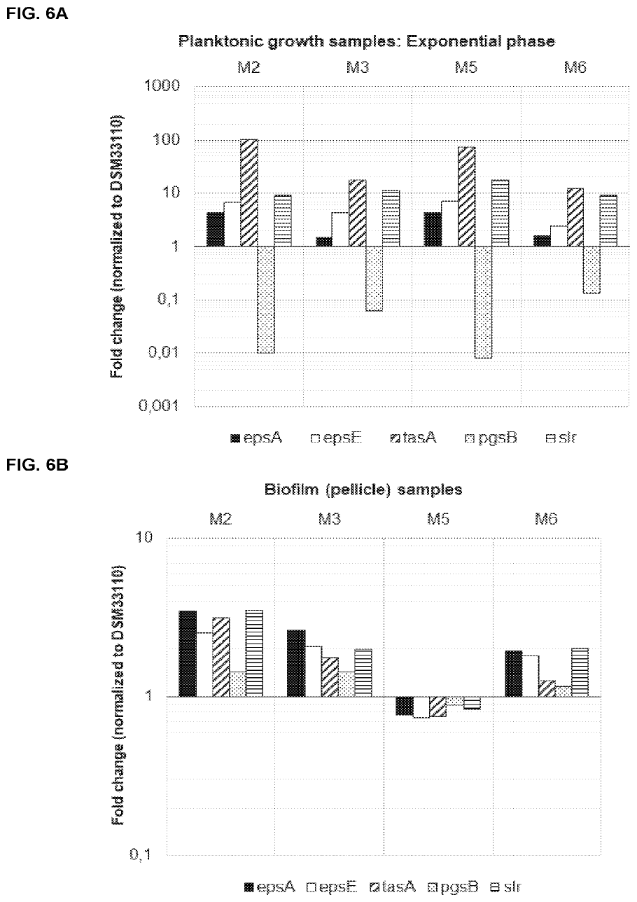

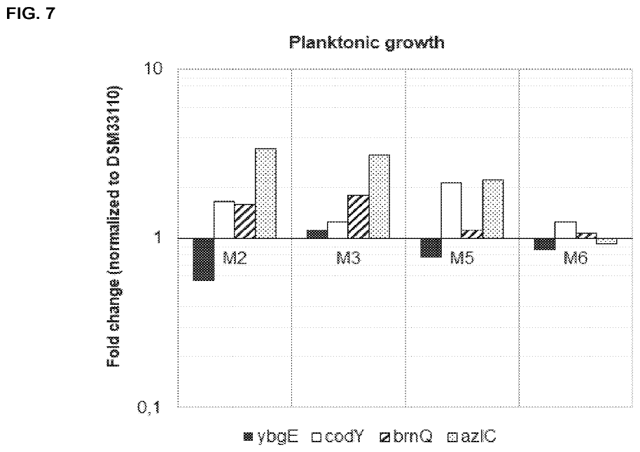

FIG. 6 shows summary of results obtained by qPCR analysis of relative gene expression levels in biofilm improved derivative strains with respect to the parental strain DSM33110. Expression levels are represented as the fold change normalized to expression in DSM33110, which value is assigned to 1. qPCR results confirmed the role of SNPs identified in genes pdeH, oppA and gntR in the regulation of biofilm formation. Single identified amino acid changes in PdeH and GntR (strains M3 and M6) trigger overexpression of Slr, a key positive regulator of biofilm formation in Bacillus, EpsA and EpsE (belonging to the EPS biosynthetic pathway), and the amyloid-like protein TasA, a biofilm matrix component (FIG. 6). A decrease in gene expression of the γ-polyglutamate operon (pgsB) was detected in all mutant backgrounds in samples collected from planktonic growing cultures, but only in DSM33115 (M5) under biofilm growth conditions. PgsB repression was particularly strong in strains DSM33112 (M2) and DSM33115 (M5), which contain a 2 amino acid deletion in the OppA oligopeptide permease, confirming the initial hypothesis linking OppA functional role to the regulation of γ-polyglutamate biosynthesis.

Expression changes analyzed in the same strains but grown in biofilm forming conditions (FIG. 6B) showed a similar relative expression pattern, however the fold changes were not so affected between the parental strain and the biofilm improved derivatives. This makes sense if we consider that under those cultivation conditions biofilm formation is induced for all strains, including the parental strain.

The involvement of GntR regulator in biofilm formation was also confirmed. GntR role could be to regulate the expression of the adjacent operon azlC-azlD-paaD-brnQ, which encodes a branched-chain amino acid transporter. Levels of branched-chain amino acids (BCAAs) regulate the activity of CodY global regulator. CodY is a global regulator, sensing intracellular BCAAs and GTP levels, and that it is present in low-GC Gram positive organisms. CodY is a pleiotropic transcriptional regulator that represses the transcription of numerous genes. As a response to lower energy and BCAA levels CodY triggers adaptation of bacterial cells by activating highly diverse mechanisms, such as secretion of proteases and the expression of amino acid transporters and catabolic pathways.

### Example 8—Quantification of Biofilm Components Associated Changes

Biofilm formation begins with the expression of matrix genes in response to some external signal (such as surfactin). Initially, cells are short, motile rods but as the biofilm develops, they form long chains of non-motile cells that adhere to each other and the surface by secreting an extracellular matrix. This extracellular matrix is essential to the integrity of the biofilm as it holds the community together. The Bacillus matrix is primarily composed of exopolysaccharide (EPS) and proteins (TasA, TapA and the hydrophobin BlsA). Several Bacillus spp. are known to produce poly-γ-glutamic acids (γ-PGA) as one of the major secreted polymeric substances. γ-Polyglutamic acid (γ-PGA) is a major component of the Bacillus biofilm matrix. Poly-γ-glutamate (PGA) is an unusual anionic polypeptide in which glutamate is polymerized via γ-amide linkages. It is water soluble, and possess good absorbability and high metal-binding capacity (Hsueh Yi-Huang 2017). In B. subtilis strains, the regulation of γ-PGA production and its physiological role are still unclear. Herein it is proposed that γ-PGA may contribute to robustness and complex morphology of the colony biofilms, suggesting a role of γ-PGA in biofilm formation. It is also suggested that γ-PGA may play a role in root colonization, pinpointing a possible function of γ-PGA in Bacilli-plant interactions. Several pathways co-regulate both γ-PGA and biofilm matrix components biosynthesis in B. subtilis, but in an opposing fashion.

γ-Polyglutamic acid (γ-PGA) produced by Bacillus strains can be detected by SDS-polyacrylamide gel electrophoresis (SDS-PAGE) and basic dye staining (Yamaguchi et al., 96). First γ-PGA was purified from supernatant samples of planktonic and biofilm growing cultures corresponding to the parental strain and biofilm improved derivatives (M2, M3, M5 and M6). γ-PGA was purified by using ethanol precipitation as described in Yamaguchi et al., 96 and samples were subjected or not to acid hydrolysis with sulfuric acid. γ-PGA purified samples were run on SDS-PAGE gels and stained with methylene blue solution. After distaining, gels were imaged and band quantification was done with Image J software.

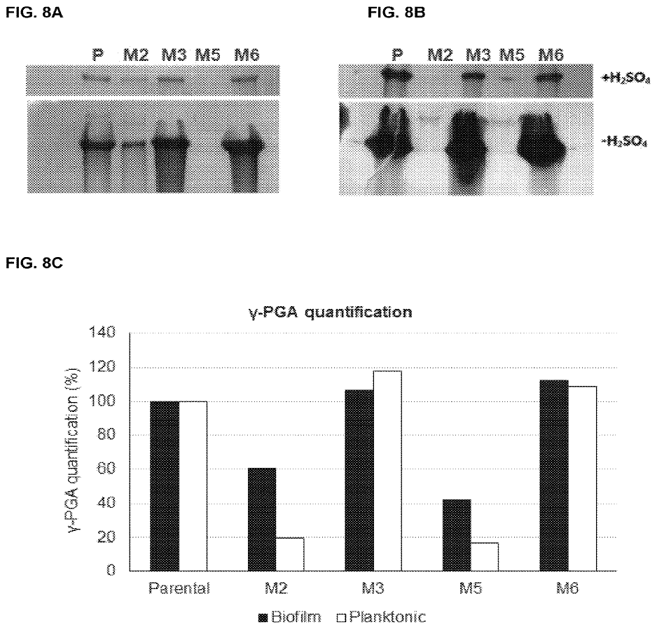

FIG. 8 corresponds to the results obtained after γ-PGA purification and quantification. Our results clearly show a decreased level of γ-PGA produced by derivative strains M2 and M5 compared to the parental strains and to the other two biofilm improved derivatives (DSM33110 and the other two derivatives M3 and M6, respectively). M2 and M5 derivatives, carrying a deletion in oppA generate drier colonies (FIG. 3), which was indicative of a defect in γ-PGA production. The OppA protein, periplasmic protein of ABC transporter Opp, is involved in regulating γ-PGA production in B. subtilis strains. These results confirm an analogous role for this protein in B. paralicheniformis.

### Example 9—Experimental Setup to Evaluate Plant Growth Promotion in Different Plant Species and Soil Types

First, plant growth promotion efficiency was compared between derivative strains using A. thaliana plants in a soil system. Experiments were performed in the plant growth chambers. A. thaliana seedlings were germinated and pre-grown in potting soil for 7 days prior inoculation. Bacterial cultures were grown over-night the day before inoculation. In the morning of inoculation day, bacterial cultures were diluted and grown to OD₆₀₀ 1 in LB broth. Bacterial cells were then washed and resuspended into 10 mM MgSO₄ buffer at OD₆₀₀ 0.1. Plants were inoculated with 1e7 CFUs (18 plant replicates per strain), comparing the results from control non-inoculated plants with the parental strain and biofilm improved derivatives. Plants grew in growth chambers for 2 weeks, with 16 h light/8 h dark photoperiod, before data collection.

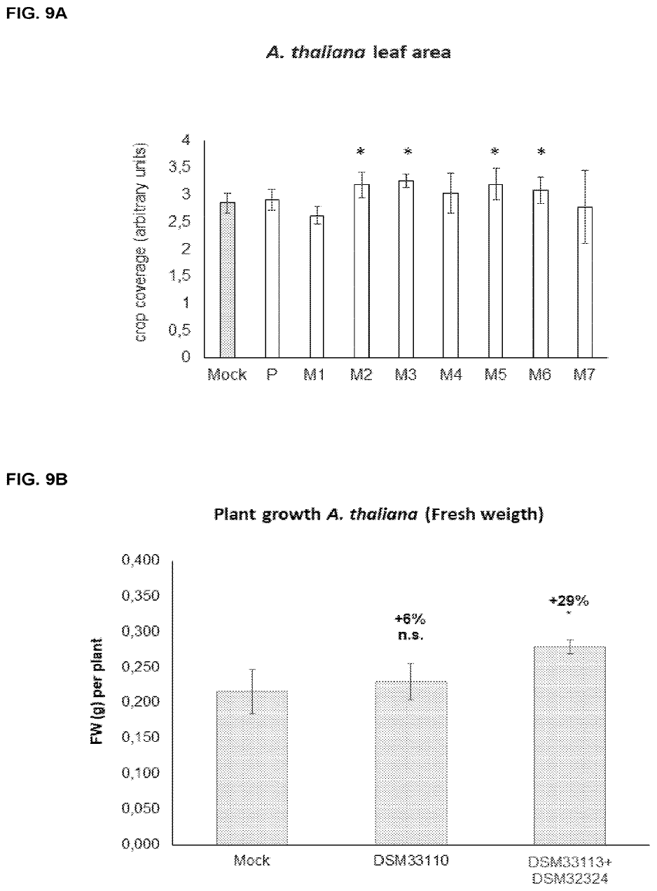

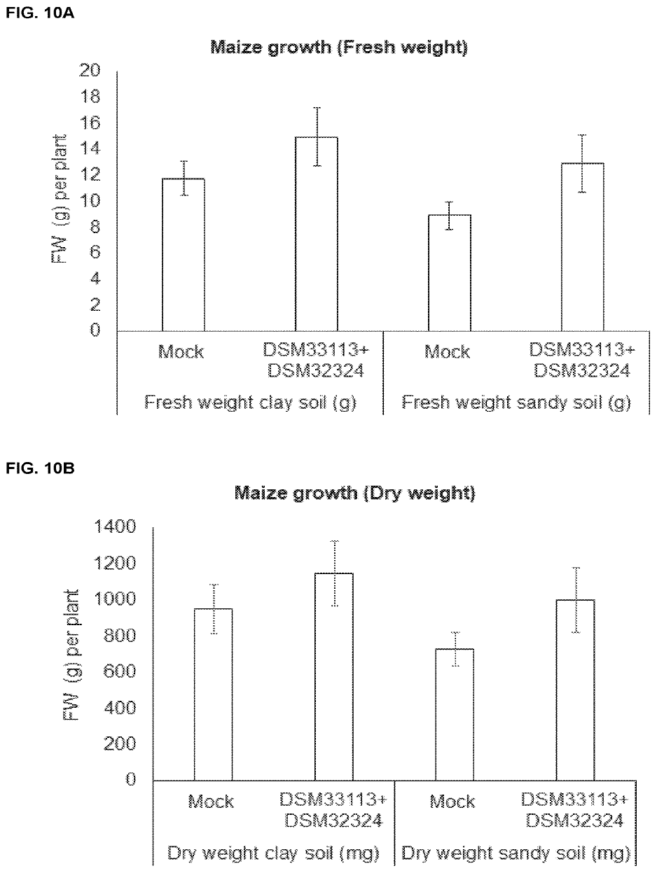

After 2 weeks, crop coverage and additional enzymatic parameters were quantified and compared between treatments (FIG. 9). Strains iPH2970 M2, M3, M5 and M6 (DSM33112, DSM33113, DSM33115 and DSM33116, respectively) showed the best crop coverage results in A. thaliana plants, showing an increase in crop coverage. Subsequent testing of the derivatives performance was done in different plant species, individually or in combination with other Bacillus strains, preferably with other B. subtilis strains.

In a second set of experiments, a different strain combination containing biofilm improved derivative M3 was compared with the original strain in their ability to promote A. thaliana plant growth. Strain B. paralicheniformis DSM33113 (M3) was co-inoculated with a Bacillus strain, preferably a Bacillus subtilis strain, as indicated in FIG. 9B. Growth conditions and experimental setup was identical to the one described above with the only exception of decreasing the number of bacterial cells inoculated. Bacterial inoculation material was prepared at OD₆₀₀ 0.01, corresponding to 1e6 CFUs/ml. Shoot fresh weight quantification was done 2 weeks post-inoculation. Results obtained showed an increase in plant growth for all treatments compared to non-inoculated plants, with a more relevant increase being observed for the combination containing the biofilm improved derivative M3 (29% increase). The increase in fresh weight per plant observed is statistically significant compared to non-inoculated plants. Therefore, strain combination DSM33113 (M3) plus B. subtilis, such as B. subtilis DSM32324 was selected to carry on additional testing in corn plants.

Maize seeds were sown in field soil collected in Taastrup, Denmark and germinated for 2 weeks in a growth chamber with 16 h light/8 h dark photoperiod at a 24-20° C. day/night temperature, prior inoculation. Bacterial cultures were grown over-night the day before inoculation. In the morning of inoculation day, bacterial cultures were diluted and grown to OD₆₀₀ 1 in LB broth. Bacterial cells were then washed and resuspended into 10 mM MgSO₄ buffer at OD₆₀₀ 0.1. Germinated seedlings (8 plant replicates per condition) were root-dipped in the bacterial solution and replanted in pots containing two types of field soil (clay and sandy soil types). Plants grew in growth chambers for 2 weeks, with 16 h light/8 h dark photoperiod, before data collection. Shoot fresh weight was recorded immediately after harvesting. The harvested samples were placed in a 70° C. degrees oven for 3 days before dry weight values were recorded.

Comparing the results from control non-inoculated plants (mock) with inoculated plants revealed a clear plant growth promotion effect in maize plants. Both positive results in clay and sandy field soil types were statistically significant, and supported the beneficial effect of the biofilm improved strain DSM33113 in combination with another Bacillus strain such as a B. subtilis strain, preferably in combination with B. subtilis DSM32324 strain.

### Conclusion

To summarize, derivative B. paralicheniformis (DSM33110) strains with improved biofilm formation were developed following an adaptive laboratory evolution campaign. Several Different derivatives were selected, and their genomes sequenced to identify the acquired genotypic changes. Derivative strains were characterized physiologically and tested for performance in plant growth experiments.

Based on our results, specific mechanisms to explain the observed phenotypic differences with the parental strain is presented.

In addition, this evolution experiments have supplied/contributed with a new Bacillus strain showing improved properties in plant growth promotion that are currently being tested as a formulated product in field trials.

### Deposits and Expert Solution

The applicant requests that a sample of the deposited microorganisms stated below may only be made available to an expert, subject to provisions governed by the Industrial Property Office, until the date on which the patent is granted.

The applicant deposited the Bacillus paralicheniformis parent strain as well as derived strains on May 8, 2019 at Deutsche Sammlung von Mikroorganismen und Zellkulturen GmbH, DSMZ, Inhoffenstr. 7B, D-38124 Braunschweig (DSMZ) and given the accession numbers:
Bacillus paralicheniformis parent strain=DSM 33110
Bacillus paralicheniformis CHCC31894=DSM 33111
Bacillus paralicheniformis CHCC31895=DSM 33112
Bacillus paralicheniformis CHCC31896=DSM 33113
Bacillus paralicheniformis CHCC31897=DSM 33114
Bacillus paralicheniformis CHCC31898=DSM 33115
Bacillus paralicheniformis CHCC31899=DSM 33116
Bacillus paralicheniformis CHCC31900=DSM 33117

The applicant deposited the Bacillus subtilis strain on Jun. 8, 2016 at Deutsche Sammlung von Mikroorganismen und Zellkulturen GmbH, DSMZ, Inhoffenstr. 7B, D-38124 Braunschweig and given the accession number: DSM32324.

The deposits were made according to the Budapest treaty on the international recognition of the deposit of microorganisms for the purposes of patent procedure.

SEQUENCES
SEQ ID NO: 1
ATGAGGGTATTCGTTGCCAGACAGCCAATATTCAACAGAAAAGAACAAGTIGTCGC
ATACGAGCTTTTATACAGAGAAAGCGAGAAAAACTTTTTTTCCGGTATTGACGGTGA
TCAAGCGACAACAGAATTGATGATTAACAGCTTTTTAAACATTGGAATCGATAAATT
GACAGAAGGCAAAAGGTATTACGTGAATTTTACCGAAGGTCTTTTGGCATCAGGGCT
GCCGACTTATTTTGATCCGGACCAGCTCGTTGTAGAAATCCTTGAAGATGTGCCGAT
CACCCCTGAGCTTATCGAAAGGTGCAGGCATTTGAAAAGCCTCGGCTACACGATTGC
CCTTGATGATTTTTGTTTAAAACATCGCGTGGAGAGGGACTTGCTCCATCAGCTTTTG
GCATCCATTGATATTTTGAAGATCGATTTTTTCAAAACGACGCGGCAGGAAAGACAG
AGCATCCTTCGATCATGCCGGAACCACCGTCTGACGTTTCTCGCTGAAAAAGTGGAG
ACGCGAAAGGATTATGAGCAGGCGGCAAAAGACGGATTTCAACTGTTTCAAGGCTA
TTTCTTCAGCGAGCCCGCTGTGATTGACGGTCAGGACATTACATATCATTTCCATGCT
TACTATGAGCTGCTGCATGAATTAAGCGAAGATCAGCCTGATATTGAAAACGTCACA
AATATTATAGAACGCGATTTGTCGCTTTCCTACCAGCTGCTGAAGCTGTTGAACTCTC
CGGCCAACCGGCCGATTCAAAAGATTAAAAGCATCCGCCAGGCTATCGTGCTTCTCG
GATTTAAAGAAATCAAAAGGTGGATCTTCATCCTCTCATTTAAGGATTTAACGAAAA
AACAAAACTCCAGCAAGAACGAAGTCGTCAAGATTTCGCTGATCCGCGCGAAGCTT
TGCGAATTGCTGGCGAAAAAAACGAATCGCCCTCAGCCGGCTTCTTATATGCTGACA
GGCATGTTTTCATTTATCGATACACTGCTTCACAAAGAACTGGCGGAAGTCATCAGC
GAACTGCCGCTGACGGACGAAGTAGGACAAGCACTGCTCGGCAAAGAAAACGATTA
TCGAAAAATCTTACGGCTTGCAAAATCGATCGAGCGAAACGAATGGGAAGACAGCA
CGCCAGAAACAGAAGGCTTAACAAAAGATGAAGCGTATCAATGCTATCTTGAAGCC
GTTGAT
SEQ ID NO: 2
MRVFVARQPIFNRKEQVVAYELLYRESEKNFFSGIDGDQATTELMINSFLNIGIDKLTEG
KRYYVNFTEGLLASGLPTYFDPDQLVVEILEDVPITPELIERCRHLKSLGYTIALDDFCLK
HRVERDLLHQLLASIDILKIDFFKTTRQERQSILRSCRNHRLTFLAEKVETRKDYEQAAK
DGFQLFQGYFFSEPAVIDGQDITYHFHAYYELLHELSEDQPDIENVTNIIERDLSLSYQLL
KLLNSPANRPIQKIKSIRQAIVLLGFKEIKRWIFILSFKDLTKKQNSSKNEVVKISLIRAKLC
ELLAKKTNRPQPASYMLTGMFSFIDTLLHKELAEVISELPLTDEVGQALLGKENDYRKIL
RLAKSIERNEWEDSTPETEGLTKDEAYQCYLEAVDWCQKLL
SEQ ID NO: 3
ATGAATAAACGCAAAACAGGATTTTCAATTTTAAGCTTGCTGCTGATCTTATCGATT
TTTCTAACGGCCTGCAACAGCGGCGAAGTCGGAGGGGACGAAAAGGAAGGCAAATC
TGACGGGAAGCCGCAGCAGGGCGGGGATCTGATTGCTGGATCCACAGGTGAACCGA
CGCTGTTTAATTCACTGTATTCAACTGATACAGCAAGTTCAGATATTGAAAGACTGA
TCTACAACACTTTGTTAGACGTTAATGAGAAACTTGAAGTGGAGAACCAGCTTGCTG
AGGAAGTAAAAGAATCGGAAGATGGTTTAACATTTGATGTTAAGCTCAAGGAAGGC
GTCAAGTTTCATGACGGTGAAGAAATGACAGCTGATGATGTTGTATTTACTTACAGC
ATTCCGATGAGTGATGACTACGTCGGAGAACGCGGCTCGAACTTTAAAATGATAGA
GTCTGTCACGAAAAAAGGTAAATATGAAGTACAGTTTAAATTAAAGAAGCCTGATC
CGTATTTTTACAATGTTACACTTGCCAGTTACGGTATTCTGCCTAAGCACATTTTAAA
AGATGTCCCAATCAGCAAACTCGGTGAACACGAATTCAATCGAAAGAATCCGATTG
GAACAGGACCGTTTAAATTTAAAGAATGGAAAGAAGGACAATATGTAAAGGTTGAG
GCTTTTGATGATTATTATGCCGGGCGTCCTCATTTAGATTCGATCACGTATAAAATTA
TTCCGGATTCAAATGCAGCGCTGTCACAGCTGCAAGCTGGAGATGTCGATTACTTGG
TCGTTACACCAGGACCAGACTATAAAACGGCCGAGAAATTTAACAATGTGAAGATG
GAAACCGATTTAGGTTTGAATTATACGTATATCGGCTGGAACGAAAGAAATGAGCT
GTTTAAGGATAAAAAGGTTCGCCAAGCGCTGACACATGCGCTTGACCGCCAGGCGC
TCGTTGACCAAGTTCTAGATGGAGATGGGGAAATCGCGAACATCCCGGAAAGCCCG
CTTTCATGGAACTATCCGGATAACAAAGATAAGTTTAAAACGTTTGAATACGATCCA
GAGAAAGCAAAAAAACTGCTCAAAGAAGCTGGATGGACAGACTCAGATGGTGATG
GGATTTTAGATAAGGACGGCAAAAAGTTTTCTTTCGTTATTAAAACGAACCAAGGAA
ACAAAACACGTGAAGACCTCGCGGTTGTTGTTCAGCAACAATTAAAAGAAATTGGT
ATTCAAGCAAAACCGCAGATTGTTGAATGGAGTGCTTTAATTGAACAAATGAATCCG
CCGAATTGGGATTTTGATGCGATGATCATGGGCTGGAGCCTTGCTACGTTCCCTGAT
CAGAGCAACATTTTCCATTCGAAAGAAGCTGAAAAAGGACTAAACTATGTTTGGTAT
CAAAATAAAAAGCTTGATAAATTGCTGGATGAAGCTAAAACGTTGAAGGATCGTGA
AGAATATAAAAAGGCGTATGAAGATATTTACGAAATTCTGGCGGAAGATCAGCCAT
ATACATTCTTGTATTATACGAACTACCATAGAGCGATGCCAAAAAATATGAAAGGTT
ACGTCTTCCATCCGAAAGAAGATTTCTATAAAGCCGAAGACTGGTGGCTGGATCAA
AAATAA
SEQ ID NO: 4
MNKRKTGFSILSLLLILSIFLTACNSGEVGGDEKEGKSDGKPQQGGDLIAGSTGEPTLFNS
LYSTDTASSDIERLIYNTLLDVNEKLEVENQLAEEVKESEDGLTFDVKLKEGVKFHDGEE
MTADDVVFTYSIPMSDDYVGERGSNFKMIESVTKKGKYEVQFKLKKPDPYFYNVTLAS
YGILPKHILKDVPISKLGEHEFNRKNPIGTGPFKFKEWKEGQYVKVEAFDDYYAGRPHL
DSITYKIIPDSNAALSQLQAGDVDYLVVTPGPDYKTAEKFNNVKMETDLGLNYTYIGWN
ERNELFKDKKVRQALTHALDRQALVDQVLDGDGEIANIPESPLSWNYPDNKDKFKTFE
YDPEKAKKLLKEAGWTDSDGDGILDKDGKKFSFVIKTNQGNKTREDLAVVVQQQLKEI
GIQAKPQIVEWSALIEQMNPPNWDFDAMIMGWSLATFPDQSNIFHSKEAEKGLNYVWY
QNKKLDKLLDEAKTLKDREEYKKAYEDIYEILAEDQPYTFLYYTNYHRAMPKNMKGY
VFHPKEDFYKAEDWWLDQK
SEQ ID NO: 5
ATGCCAGTCAATTCGTTTGACAACTATCCAATGTCTTGGAAACCTGATAAGAAAGCA
TTGAAGCGTCCTTATTATTATTCGATTGCGACATTGCTTGAAGAGGATATCGTAAAC
GGTTTTTTGGCGCCTGGGACAAAGCTGCCTCCGCAACGGGAACTGGCAGATTTTCTT
GATTTAAACTTTACCACGATTACACGCGCCTACAAACTATGTGAGTTCAAGGGGCTG
ATTTATGCTGTCACCGGAAGCGGCACCTTCGTCGCTCCTAATGCTGCCCGCTCTATC
ACCATTTCCGCAGATAAGGTGACAAACTGCATTGATCTCGGATTTGTAGCCTCTTTT
GAGCAAACCAACGGAATGGTAGCGGAGGTTGTTCAAAAAGCTGCAGATAAAAGCTA
TTTGGAGAAGCTAATGGACTACAATGACCCGACTGGTATTCCGCATCAAAAAACGG
CAGGGCTAAACTGGATGGAATCTTTCGGTATTCACGCAGACCAAGAACATATTGCG
ATTGTTTCCGGTGCTCAAAATGCGTTGGCCATTGCATTGACCTCGCTGTTTGACCCTG
GTGACCGCATTGCAACTGACCTATACACGTATTCGAACTTTATTGAGCTGGCCAAAA
TGCTCCATATTCAATTGGTACCTGTTTCCGGTGACCAGTATGGAATGCTGCCGGATG
AACTTGAAAAGCAGTGCTGCCAGACGAAAATTCACGGCATATTTCTGATGCCCTCAT
GCTGTAATCCGACAACCGTGATGATATCAGATGTTCGAAAGCATGAATTAGTGGAG
GCCATCCGCAAGCATGATTTGCTGTTGATCGAGGATGATATTCATGCGTTTCTGACG
GCAGGGATTGTATCAGATTATCAGCAGCCAATGTTTAGTTTGCTTCCAGATCAGAGC
ATATACATTTGCAGCACCTCAAAGTCGATATGCTCCGGGTTAAGAGTTGCCTATATG
GTGTATGGGGATGCTTTACGGGAAAAGATATTGCAGGGCATTTTTAACATCAATGTC
AAAACGTCATCTTTAGATGCGGAGGTCATTACTGAGCTGATTTTATCAGGTAAGGCT
CATGAAATCGTTGCTCAAAAGAAAAAGCTTGCACAGTCGGCCAATGATCTTTATGCG
GCATATTTTCCTGTAACTGAGCCTGGTGAACATCCTCTTAGTTTATACCGATGGCTTC
CGATTGAAGAGCATGCTGACTCATCACAATTGGAGACGGATTTGAGGAAGCGTGGG
ATTCGGGTTTTTCATTCCGACCGTTTTCTCAGCGGGCAGACGACGCGCGAAAAATAT
TTGCGCATTGCGCTTTCTTCTACAAATTCATTAGATGAGCTGAAATTAGGGTTGGAT
ATATTAAAACAGTATCTCGGATAA
SEQ ID NO: 6
MPVNSFDNYPMSWKPDKKALKRPYYYSIATLLEEDIVNGFLAPGTKLPPQRELADFLDL
NFTTITRAYKLCEFKGLIYAVTGSGTFVAPNAARSITISADKVTNCIDLGFVASFEQTNGM
VAEVVQKAADKSYLEKLMDYNDPTGIPHQKTAGLNWMESFGIHADQEHIAIVSGAQNA
LAIALTSLFDPGDRIATDLYTYSNFIELAKMLHIQLVPVSGDQYGMLPDELEKQCCQTKI
HGIFLMPSCCNPTTVMISDVRKHELVEAIRKHDLLLIEDDIHAFLTAGIVSDYQQPMFSLL
PDQSIYICSTSKSICSGLRVAYMVYGDALREKILQGIFNINVKTSSLDAEVITELILSGKAH
EIVAQKKKLAQSANDLYAAYFPVTEPGEHPLSLYRWLPIEEHADSSQLETDLRKRGIRVF
HSDRFLSGQTTREKYLRIALSSTNSLDELKLGLDILKQYLG
SEQ ID NO: 7
TTGAAGAAGCGTTTGTCATTTATCAGTTTAATGCTCATTTTCACACTCGTCCTCAGCG
CCTGCGGCTTCGGCTCAAGCTCCGGTGACGGCGGTAAAAAAGACAGCAAAGGGAAA
GACACATTAAATGTCAACATTAAAACAGAACCGTTTTCACTACATCCGGGACTCGCA
AACGATTCGGTGTCTGCAAACGTGCTTCGTCAGACTTTTGAAGGATTGACGACAATC
GGTAAAGATGGAAAGCCGGTTGAAGCGGCAGCCGAAAAAATCGAAGTCAGCGACG
ACCAAAAAACATACACATTCACGCTCCGCGACGCGAAATGGTCAAATGGAGATCCT
GTAACAGCAGAGGATTTTGAATACGCATGGAAATGGGCGCTCGACCCTAAAAACGA
ATCGCAATATGCGTATCAGCTTTACTACTTAAAAGGCGGAGAAGCAGCGAACACCG
GCAAAGGGAAAATTGAAGATGTCGGCGTTAAAGCTGTTAATGATAAGACTTTAAAA
GTCGAGCTTGAAAAACCGACACCGTATTTTACTGAACTGACAGCATTCTACACATAT
ATGCCAGTCAATAAAAAGGTAGCAGAGAAGAATGCGAAATGGTACACAAACGCAG
ATGAGAACTACGTATCTAACGGACCTTTCAAAATGGCGAAATGGAAGCACAGCGGA
AACATCGTACTGGAGAAAAACGACCAGTACTGGGATAAAGACGCTGTTAAGCTGAA
GAAAATCAATATGGCGATGGTCAACGATCCGAACACTGGTCTGAACATGTACAAAA
AAGGCGAGCTAGACTTTGTAGGACAGCCGCTTGACCAGATTTCAACGGATGCGATTC
CAAGCCTGAAAAAAGAAGGCCTGAACATTGATCCGTTCGCATCGGTTTACCTGTACA
AATTCAACACTGAAGCGGCTCCGCTGAACAATGTCAACATCCGTAAGGCGCTGACA
TACGCGATCAACCGCGAAGCGATCGTCAAAAACATCACGCAAGCGGAACAGCTGCC
TGCGATGGGATTAGTGCCGCCGGCAGTCCACGGCTTTGAGTCAAATAAAGGCTATTT
CAAAGACCATGATGTTGATAAGGCGAAAGAATACTTGGAAAAAGGTTTGAAAGAGC
TCGGTTTGAAAAAAGCGTCAGACCTTCCGAAAATCACGCTTTCCTTCAACACGGATG
AAGCACACCAAAAAATCGCTCAAGCCGTTCAGGAAATGTGGAACAAGGAACTGGGC
TTAGATGTCGAATTAGGCAATGAAGAATGGAACGTATACATCGATAAGCTCCATGC
AGGAAACTATCAAATCGGCCGTTTAGGCTGGACCGCCGACTTTAACGACGGAATGA
ACTTCCTTGAAACATACCGCGACAAAGAAGGCGGAAACAATGATACGAACTGGGAA
AACGCAAAGTATAAAGAGCTGCTGAATAAAGCATCGAGAGAAACTGATTCAGCGAA
ACGCATCGAGCTGATGAAAGAAGCAGAAAGCATCATCATGGATGAGCTGCCGGTTG
CACCGATCTACTTCTACACAATGCCGTATCTTCATGATGAAAGCTTAAAAGACTTTG
TTCTAACTGGTACTGGTGAGATCTATTTCAAAACCGCGCATTTTGAATAA
SEQ ID NO: 8
MKKRLSFISLMLIFTLVLSACGFGSSSGDGGKKDSKGKDTLNVNIKTEPFSLHPGLANDS
VSANVLRQTFEGLTTIGKDGKPVEAAAEKIEVSDDQKTYTFTLRDAKWSNGDPVTAEDF
EYAWKWALDPKNESQYAYQLYYLKGGEAANTGKGKIEDVGVKAVNDKTLKVELEKP
TPYFTELTAFYTYMPVNKKVAEKNAKWYTNADENYVSNGPFKMAKWKHSGNIVLEKN
DQYWDKDAVKLKKINMAMVNDPNTGLNMYKKGELDFVGQPLDQISTDAIPSLKKEGL
NIDPFASVYLYKFNTEAAPLNNVNIRKALTYAINREAIVKNITQAEQLPAMGLVPPAVHG
FESNKGYFKDHDVDKAKEYLEKGLKELGLKKASDLPKITLSFNTDEAHQKIAQAVQEM
WNKELGLDVELGNEEWNVYIDKLHAGNYQIGRLGWTADFNDGMNFLETYRDKEGGN
NDTNWENAKYKELLNKASRETDSAKRIELMKEAESIIMDELPVAPIYFYTMPYLHDESL
KDFVLTGTGEIYFKTAHFE

### REFERENCES

Kampf J, Stülke J. (2017) Cyclic-di-GMP signalling meets extracellular polysaccharide synthesis in Bacillus subtilis. Environ Microbiol Rep. Jun; 9 (3): 182-185. doi: 10.1111/1758-2229.12530. Epub 2017 Apr. 3
Chen, Y., Chai, Y., Guo, J. H. and Losick, R. (2012) Evidence for cyclic Di-GMP-mediated signaling in Bacillus subtilis. J Bacteriol, 194, 5080-5090
Comella, N. and Grossman, A. D. (2005) Conservation of genes and processes controlled by the quorum response in bacteria: characterization of genes controlled by the quorum-sensing transcription factor ComA in Bacillus subtilis. Mol Microbiol, 57, 1159-1174
Stanley, N. R. and Lazazzera, B. A. (2005) Defining the genetic differences between wild and domestic strains of Bacillus subtilis that affect poly-gamma-dl-glutamic acid production and biofilm formation. Mol Microbiol, 57, 1143-1158
Romero, D., Aguilar, C., Losick, R. and Kolter, R. (2010) Amyloid fibers provide structural integrity to Bacillus subtilis biofilms. Proc Natl Acad Sci USA, 107, 2230-2234
Hsueh, Y. H., Huang, K. Y., Kunene, S. C. and Lee, T. Y. (2017) Poly-gamma-glutamic Acid Synthesis, Gene Regulation, Phylogenetic Relationships, and Role in Fermentation. Int J Mol Sci, 18
Yamaguchi, F., Ogawa, Y., Kikuchi, M., Yuasa, K. and Motai, H. (1996) Detection of gamma-Polyglutamic Acid (gamma-PGA) by SDS-Page. Biosci Biotechnol Biochem, 60, 255-258
EP 0705807
BR PI 0604602-9
Siddiqui and Mahmood (1999). Bioresource Technology 69; 167-179

1. A method comprising applying a Bacillus to one or more of a plant propagation material, a plant and a plant growth medium, said Bacillus having a loss-of-function mutation in its closest ortholog of the pdeH gene found in Bacillus paralicheniformis DSM 33110.

2. The method of claim 1, wherein said closest ortholog of the pdeH gene found in Bacillus paralicheniformis DSM 33110 is at least 95% identical to and less than 100% identical to the sequence set forth herein as SEQ ID NO: 1.

3. The method of claim 1, wherein said Bacillus further comprises a mutation in its closest ortholog of the oppA gene found in Bacillus paralicheniformis DSM 33110.

4. The method of claim 3, wherein said closest ortholog of the oppA gene found in Bacillus paralicheniformis DSM 33110 is at least 95% identical to and less than 100% identical to the sequence set forth herein as SEQ ID NO: 3 and/or the to the sequence set forth herein as SEQ ID NO: 7.

5. The method of claim 1, wherein said Bacillus further comprises a mutation in its closest ortholog of the gntR gene found in Bacillus paralicheniformis DSM 33110.

6. The method of claim 5, wherein said closest ortholog of the gntR gene found in Bacillus paralicheniformis DSM 33110 is at least 95% identical to and less than 100% identical to the sequence set forth herein as SEQ ID NO: 5.

7. The method claim 1, wherein said Bacillus has a genome that is at least 95% identical to the genome of Bacillus paralicheniformis DSM 33110.

8. The method claim 1, wherein said Bacillus has a genome that is at least 97% identical to one or more of Bacillus paralicheniformis DSM 33111, Bacillus paraicheniformis DSM 33112, Bacillus paralicheniformis DSM 33113, Bacillus paralicheniformis DSM 33114, Bacillus paralicheniformis DSM 33115, Bacillus paralicheniformis DSM 33116 and Bacillus paralicheniformis DSM 33117.

9. The method claim 1, wherein said Bacillus is Bacillus paralicheniformis DSM 33111, Bacillus paraicheniformis DSM 33112, Bacillus paralicheniformis DSM 33113, Bacillus paralicheniformis DSM 33114, Bacillus paralicheniformis DSM 33115, Bacillus paralicheniformis DSM 33116 or Bacillus paralicheniformis DSM 33117.

10. The method of claim 1, wherein said Bacillus is applied to a plant propagation material.

11. The method of claim 1, wherein said Bacillus is applied to a living plant.

12. The method of claim 1, wherein said Bacillus is applied to a plant growth medium.

13. The method of claim 1, wherein said Bacillus is applied as part of a composition comprising one or more biostimulants, fungicides, herbicides, insecticides, nematicides, nutrients and/or plant growth regulators.

14. The method of claim 1, wherein said method further comprises applying Bacillus subtilis DSM 32324 to said one or more of a plant propagation material, a plant and a plant growth medium.

15. The method of claim 1, wherein said Bacillus is applied as part of a composition comprising Bacillus subtilis DSM 32324.
